(19) United States
(12) Patent Application Publication
(10) Pub. No.: US 2015/0183846 A1
(43) Pub. Date: Jul. 2, 2015

(54) HUMAN FUSION PROTEINS COMPRISING SINGLE CHAIN TNFALPHA AND TARGETING DOMAINS

(71) Applicant: Scil Proteins GmbH, Halle/Saale (DE)

(72) Inventors: Christian Lange, Halle (DE); Manja Gloser, Teutschenthal (DE); Susan Lorey, Halle (DE); Markus Fiedler, Halle/Saale (DE)

(73) Assignee: Scil Proteins GmbH, Halle/Saale (DE)

(21) Appl. No.: 14/407,213

(22) PCT Filed: Jun. 13, 2013
(86) PCT No.: PCT/EP2013/062310

§ 371 Date: Dec. 11, 2014

(30) Foreign Application Priority Data
Jun. 13, 2012 (EP) 12171789.6

(51) Int. Cl. C07K 14/525; A61K 38/19; A61K 38/17; C07K 14/47

(52) U.S. Cl. CPC C07K 14/525; C07K 14/47; A61K 38/191; A61K 38/1709; C07K 2319/33; A61K 38/00

(57) ABSTRACT

The present invention relates to fusion proteins in which a biologically active moiety is linked to a targeting domain. The invention specifically concerns fusion proteins comprising single-chain (sc) TNFalpha monomers as biologically active moiety and a specific targeting domain, preferably fusion proteins comprising at least three scTNFalpha monomers and modified hetero-dimeric ubiquitin proteins with high affinity to target molecules (Affilin® molecules). The invention further relates to these fusion proteins for use in medicine, in particular in the treatment of cancer. The invention is further directed to pharmaceutical compositions comprising such fusion proteins in combination with chemotherapeutics agents.

The present invention relates to fusion proteins comprising a biologically active single chain TNFalpha molecule that comprises at least three TNFalpha monomers joined by linkers and a targeting domain capable of binding to a target with high specific binding affinity. For example, a modified hetero-dimeric ubiquitin protein (Affilin®) can be used as specific targeting domain, e.g. as tumor targeting domain. The invention further relates to these fusion proteins for use in medicine, in particular for use in the treatment of cancer. The invention provides polynucleotides encoding such fusion proteins, vectors comprising such polynucleotides, and host cells comprising these fusion proteins, polynucleotides, or vectors. The invention is also directed to pharmaceutical compositions comprising a pharmaceutically acceptable carrier in combination with such fusion proteins, optionally in combination with a chemotherapeutic agent. Further, combination therapies of such fusion proteins with chemotherapeutic substances are provided. Moreover, the invention relates to a method for the generation of said fusion proteins with single chain TNFalpha molecule and specific targeting domain.

### BACKGROUND OF THE INVENTION

There is a growing demand for binding molecules consisting of amino acids which are not immunoglobulins. While until now antibodies represent the best-established class of binding molecules there is still a need for new binding molecules in order to target ligands with high affinity and specificity since immunoglobulin molecules suffer from major drawbacks. Although antibodies can be produced quite easily and may be directed to almost any target, they have a quite complex molecular structure. There is an ongoing need to substitute antibodies by smaller molecules which can be handled in an easy way. These alternative binding agents can be beneficially used for instance in the medical fields of diagnosis, prophylaxis and treatment of diseases.

Small proteins having relatively defined 3-dimensional structures, commonly referred to as protein scaffolds, may be used as starting material for the design of said alternative binding agents. These scaffolds typically contain one or more regions which are amenable to specific or random sequence variation, and such sequence randomization is often carried out to produce a library of proteins from which the specific binding molecules may be selected. Molecules with a smaller size than antibodies and a comparable or even better affinity towards a target antigen are expected to be superior to antibodies in terms of pharmacokinetic properties and immunogenicity.

### Specific Targeting Proteins Based on Modified Ubiquitin

Ubiquitin is a small, monomeric, and cytosolic protein which is highly conserved in sequence and is present in all known eukaryotic cells from protozoans to vertebrates. The polypeptide chain of ubiquitin (see SEQ ID NO: 1) consists of 76 amino acids folded in an extraordinarily compact α/β structure (Vijay-Kumar et al., 1987 Apr. 5; J. Mol. Biol., 194(3):531-44). Almost 87% of the polypeptide chain is involved in the formation of the secondary structural elements by means of hydrogen bonds. Secondary structures are three and a half alpha-helical turns as well as an antiparallel β sheet consisting of four strands. A further structural feature is a marked hydrophobic region in the protein interior between the alpha helix and the β sheet.

Because of its small and compact size, artificial preparation of ubiquitin can be carried out both by chemical synthesis and by means of biotechnological methods. Due to the favourable folding properties, ubiquitin can be produced by genetic engineering using microorganisms such as Escherichia coli in high yields either in the cytosol or in the periplasmic space. Ubiquitin is only 1/10 the size of a conventional antibody allowing pharmacokinetic properties complementary to antibodies.

Affilin® molecules are created by engineering de-novo binding sites on the surface of the human serum protein Ubiquitin (WO 04/106368; Affilin® is registered trademark of Scil Proteins GmbH). Ubiquitin can be multimerized to generate high specific binding molecules with high target affinity (WO 2011/073214). De-novo binding sites are generated by randomization of up to 15 surface-exposed amino acids, generating large libraries which comprise more than 10¹⁰ different Affilin® molecules—each individual variant with specific binding and physico-chemical characteristics.

Affilin molecules combine the high target affinity and specificity of antibodies with beneficial features of small molecules including small size, high stability, cost effective manufacturing as well as ease of chemical or genetic manipulation. The structural similarity to Ubiquitin does offer many advantages over other scaffold molecules, like low risk of immunogenicity and rapid preclinical development without the need for surrogates. The combination of these advantages makes Affilin molecules particularly attractive for the development of biopharmaceutical drugs.

### Extra-Domain B of Fibronectin as Tumor Specific Protein

The extra-domain B (ED-B) of fibronectin represents one of the most selective markers associated with angiogenesis and tissue remodeling. It is abundantly expressed around new blood vessels, but undetectable in virtually all normal adult tissues (except for uterus and ovaries). ED-B is known to be involved primarily in cancer. High levels of ED-B expression were detected in primary lesions as well as metastatic sites of many human solid cancer entities, including breast, non-small cell lung, colorectal, pancreatic, human skin, hepatocellular, intracraneal meningeoma, glioblastoma. In solid cancer tissues, ED-B is either detected surrounding pro-angiogenic vessels or in a mixed mode of perivascular and stromal expression (Menrad and Menssen, 2005 Expert Opin Ther Targets 9:491-500). Furthermore, ED-B can be bound to diagnostic agents and used as diagnostic tool. One example is its use in molecular imaging of atherosclerotic plaques and detection of cancer, for example by immunoscintigraphy of cancer patients. Plenty of additional diagnostic uses are conceivable.

The extra-domain B (ED-B) of fibronectin is a small domain which is inserted by alternative splicing of the primary RNA transcript into fibronectin. Fibronectins are high molecular weight extracellular matrix glycoproteins abundantly expressed in healthy tissues and body fluids. The ED-B molecule is either present or omitted in fibronectin molecules of the extracellular matrix.

The amino acid sequence of 91 amino acids of human extra-domain B (ED-B) of fibronectin is shown in SEQ ID NO: 2 (a start methionine has to be added to SEQ ID NO: 2 for the expression of the protein). ED-B is detected in mammals, e.g. in rodents, cattle, primates, carnivore, human etc. Examples of animals in which there is a 100% sequence identity to human ED-B are Rattus norvegicus, Bos taurus, Mus musculus, Equus caballus, Macaca mulatta, Canis lupus familiaris, and Pan troglodytes.

ED-B specifically accumulates in neo-vascular structures and represents a target for molecular intervention in cancer. A number of antibodies or antibody fragments to the ED-B domain of fibronectin are known in the art as potential therapeutics for cancer and other indications (see, for example, WO 97/45544, WO 07/054120, WO 99/58570, WO 01/62800). A human single chain Fv antibody fragment specific to the ED-B domain of fibronectin was verified to selectively target tumor neovasculature, both in experimental tumor models and in patients with cancer. Furthermore, conjugates comprising an anti-ED-B antibody or an anti-ED-B antibody fragment with IL-12, IL-2, IL-10, IL-15, IL-24, or GM-CSF have been described. In particular, such conjugates were described for targeting drugs for inhibiting diseases such as cancer, angiogenesis, or neoplastic growth (see, for example, WO 06/119897, WO 07/128563, WO 01/62298). The selective targeting of neovasculature of solid tumors with anti-ED-B antibodies or anti-ED-B antibody fragments conjugated to an appropriate effector function such as a cytotoxic or an immunostimulating agent has proven to be successful in animal experiments. For the therapy of pancreatic cancer, fusion proteins comprising an Interleukin-2 part (IL-2) and an anti-ED-B antibody part were combined with a small molecule.

WO 2011/073208 and WO 2011/073209 disclose multimeric proteins based on modified ubiquitin with high affinity binding to the extradomain B of fibronection (ED-B). The applications describe anti-ED-B binding molecules showing a highly efficient targeting of tumor vasculature.

### Native TNFalpha

TNFalpha (tumor necrosis factor alpha) is a 212 amino acid cytokine known to be part of inflammation reactions by regulating immune cells. Native TNFalpha is, among other biological functions, able to induce apoptotic cell death and inhibit tumorigenesis, therefore being an interesting and validated pharmacological protein. Further, the vascular permeability of endothelial tissues, including tumor tissues, is increased by TNFalpha. The native and biologically active TNF molecule is a non-covalently linked homo-trimeric protein.

### Fusion Proteins of Anti-ED-B Binding Proteins and Native TNFalpha

A conjugate of a tumor targeted antibody and native TNFalpha was tested clinically and failed to improve efficacy although the conjugate accumulates at the tumor site (Borsi et al., 2003 Blood 102, 4384-4392).

Conjugates of tumor-targeted Affilin® and native TNFalpha showed improved affinity and specificity compared to conjugates of antibodies with native TNFalpha (see Scil Proteins' patent applications WO 2011/073208 and WO 2011/073209).

TNFalpha occurs as non-covalently connected homo-trimer binding to two different receptors. Preclinical evidence is available for TNF to damage tumor vasculature (Balkwill, 2009, Nature Reviews 9:361-371). Reasons for the damage of tumor vasculature might be increasing endothelial permeability, inducing endothelial apoptosis and tumor specific immune response. However, native TNFalpha is not suited for systemic therapeutic application because the systemic free maximal tolerated dose of native TNFalpha is lower than the effective dose. Thus, the application of non-targeted TNFalpha leads to severe toxicity and life-threatening side effects.

Single Chain TNFalpha (scTNF)

In the prior art, single chain (sc) TNFalpha proteins of at least three monomers connected by peptide linkers are described generally. For example, Krippner-Heidenreich et al. (The Journal of Immunology, 2008, 180, 8176-8183) describe polypeptides which consist of at least three monomers of a TNF family ligand which are connected by peptide linkers. Importantly, it was shown that although this construct is less toxic than wildtype TNFalpha, it shows the same bioactivity as native TNF.

### Technical Problems Underlying the Present Invention and their Solution

Since cancer represents one of the leading causes for death worldwide, there is a growing need for improved agents for treating cancer. Current chemotherapeutic agents and radiation treatment suffer from poor selectivity due to an undirected cytotoxic mechanism of action. Most chemotherapeutic agents do not accumulate at the tumor site and thus fail to achieve adequate levels within the tumor. This results in significant side effects. Further, the toxicological profile of many chemotherapeutics limits dosing and thus the beneficial effect of the chemotherapeutics. Chemotherapeutic drugs, if given alone, often show poor tissue penetration and poor tumor uptake resulting in the accumulation of chemotherapeutic drugs in healthy tissue. Needless to say that there is a strong medical need to effectively treat cancer.

Innovative cancer treatments use tumor targeted delivery of anti-cancer drugs. These drugs should be directly targeted to the tumor and spare healthy tissue. Further, the functional component of the drugs should efficiently penetrate tumors. The prior art describes conjugates comprising a pharmaceutically active component and a binding protein (typically an antibody) which is directed against tumor antigens. However, these conjugates have drawbacks on the side of the pharmaceutically active component and/or on the side of the binding protein.

There remains a strong need in the art for efficient tumor targeted therapeutics. Ideally, innovative conjugates in which the binding protein does not have the disadvantages of commonly used antibodies and in which the pharmaceutically active component exhibits an outstanding anti-tumor activity should be efficient therapeutics. In order to achieve this, the tumor target should be highly tumor specific and abundant in tumor tissue. Binding to a tumor target should occur with high affinity and selectivity. Further, in addition to high affinity target binding a tumor therapeutic should have a highly active functional domain employing a therapeutic effect. The currently developed conjugates of a tumor targeting domain and native (wildtype) TNFalpha involve a comparable disadvantage of low in vivo stability and high systemic toxicity.

Therefore, it was an object of the present invention to provide novel fusion proteins comprising (i) a targeting domain with high affinity to a tumor target and (ii) a functional domain with an anti-tumor activity.

This object is solved by the provision of the novel fusion proteins described herein. The fusion proteins of the present invention function as tumor targeted therapeutics and comprise (i) binding proteins that are advantageous as compared to antibodies and (ii) functional components that exhibit an improved anti-tumor activity. More specifically, preferred fusion proteins of the invention comprise an ED-B-specific binding molecule and single chain human Tumor Necrosis Factor alpha (scTNFα) as targeted therapeutic, in particular for cancer treatment. The intended therapeutic effect is triggered by the enforced site-directed extravasation of a co-administered cytotoxic drug.

Although scTNFalpha proteins were described in the art (see above), fusion proteins comprising (a) linker connected TNFalpha monomers and (b) specific targeting moieties have not been described in the prior art. In particular, it was unknown and has not been tested whether the presence of an additional targeting moiety would interfere with correct trimerization of the TNFalpha monomers which is required for the biological activity of TNFalpha.

An additional advantage associated with the fusion proteins of the present invention is the enhanced efficacy of approved chemotherapeutics and the enhanced permeability of tumor tissues due to the scTNFalpha domain.

An additional advantage associated with the fusion proteins of the present invention is an enhanced stability in plasma as compared to fusion proteins consisting of targeted binding proteins and native TNFalpha.

A further advantage associated with the fusion proteins of the present invention is the increased biological half-time in the body as compared to targeted binding proteins without a scTNFα molecule. Without wishing to be bound by any particular theory, it is assumed that a reduced clearance of the fusion proteins of the invention causes the increased biological half-time.

Additionally, the systemic toxicity is anticipated to be lower compared to corresponding TNFalpha conjugates.

The above-described objects are solved and the advantages are achieved by the subject-matter of the enclosed independent claims. Preferred embodiments of the invention are included in the dependent claims as well as in the following description, examples and figures.

The above overview does not necessarily describe all problems solved by the present invention.

### SUMMARY OF THE INVENTION

In a first aspect the present invention relates to a fusion protein comprising, essentially consisting of or consisting of the following parts: (i) a biologically active single chain TNFalpha molecule that comprises at least three TNFalpha monomers joined by linkers; (ii) a targeting domain that is capable of binding to a target molecule with a specific binding affinity to the target molecule of Kd≦10⁻⁷M; and (iii) optionally a linker between (i) and (ii).

In a second aspect the present invention relates to the fusion protein according to the first aspect for use in medicine.

In a third aspect the present invention relates to the fusion protein according to the first aspect for use in the treatment of cancer.

In a fourth aspect the present invention relates to a polynucleotide encoding the fusion protein as defined in the first aspect.

In a fifth aspect the present invention relates to a vector comprising the polynucleotide of the fourth aspect.

In a sixth aspect the present invention relates to a host cell comprising: a fusion protein as defined in the first aspect; a polynucleotide as defined in the fourth aspect; or a vector as defined in the fifth aspect.

In a seventh aspect the present invention relates to the fusion protein according to the first aspect for use in medicine, wherein the fusion protein is for administration in combination with an anti-cancer (e.g. chemotherapeutic) agent.

In an eighth aspect the present invention relates to the fusion protein according to the first aspect for use in the treatment of cancer, wherein the fusion protein is for administration in combination with one or more chemotherapeutic agents.

In a ninth aspect the present invention relates to a pharmaceutical composition comprising: a fusion protein as defined in the first aspect; further comprising a pharmaceutically acceptable carrier, and optionally comprising one or more chemotherapeutic agents.

In a tenth aspect the present invention relates to a method for the preparation of a fusion protein as defined in the first aspect, said method comprising the following steps:

(a) preparing a nucleic acid encoding a fusion protein as defined in the first aspect;
(b) introducing said nucleic acid into an expression vector;
(c) introducing said expression vector into a host cell;
(d) cultivating the host cell;
(e) subjecting the host cell to culturing conditions under which a fusion protein is expressed from said vector, thereby producing a fusion protein as defined in the first aspect;
(f) optionally isolating the fusion protein produced in step (e).

In an eleventh aspect the present invention relates to a method for generation of a fusion protein as defined in the first aspect, said method comprising the following steps:

(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified;
(b) providing a target molecule as potential ligand;
(c) contacting said population of differently modified proteins with said target molecule;
(d) identifying a modified dimeric ubiquitin protein by a screening process, wherein said modified dimeric ubiquitin protein binds to said target molecule with a specific binding affinity of Kd≦10⁻⁷ M;
(e) isolating said modified dimeric ubiquitin protein with said binding affinity;
(f) identifying a polynucleotide sequence encoding the modified dimeric ubiquitin protein of step (e);
(g) preparing a nucleic acid molecule comprising in frame:

(1) the polynucleotide sequence of step (f);

(2) a polynucleotide sequence encoding a biologically active single chain TNFalpha molecule;

(3) optionally a polynucleotide sequence encoding a peptide linker, wherein this polynucleotide sequence encoding a peptide linker is positioned between the polynucleotide sequence according to (1) and the polynucleotide sequence according to (2);

(h) introducing the nucleic acid molecule prepared in step (g) into an expression vector;
(i) introducing said expression vector into a host cell;
(j) subjecting the host cell to culturing conditions under which a protein is expressed from said vector, thereby producing a fusion protein comprising the modified dimeric ubiquitin protein identified in step (d), a biologically active single chain TNFalpha molecule; and optionally a peptide linker; and
(k) optionally isolating the fusion protein produced in step (j)

This summary of the invention does not necessarily describe all features of the present invention. Other embodiments will become apparent from a review of the ensuing detailed description.

### BRIEF DESCRIPTION OF THE DRAWINGS

FIG. 1 shows an alignment of TNFalpha sequences

The alignment compares TNFalpha sequences from different species, namely human TNFalpha (TNFahuman; SEQ ID NO: 13), murine TNFalpha (TNFamouse; SEQ ID NO: 14), rat TNFalpha (TNFarat; SEQ ID NO: 15); a consensus sequence (prim. cons.; SEQ ID NO: 25) is shown. A star below the sequences shows identity between the TNFalpha sequences of different mammalian species; dots refer to similarities in the amino acids.

FIG. 2 shows fusion proteins with scTNFalpha and tumor targeting domains

The fusion protein consists of a cancer target-binding protein at the N-terminus and three monomers of TNFalpha connected by linkers at the C-terminal part of the fusion protein. The cancer target-binding part of the fusion protein is based on two differently modified monomeric ubiquitin subunits linked via a short peptide linker (GIG). The monomers that were used for substitutions are based on an ubiquitin mutein which differs from the wild-type sequence according to SEQ ID NO: 1 by three amino acid exchanges: F45W, G75A, and G76A (=SEQ ID NO: 10).

The TNFalpha sequence is shown in italics. Substitutions in the ubiquitin subunits which are required for high affinity binding to the target ED-B are highlighted by using bold-type. The amino acid exchanges F45W, G75A, and G76A are not highlighted because they are not involved in target binding. Linker regions are underlined.

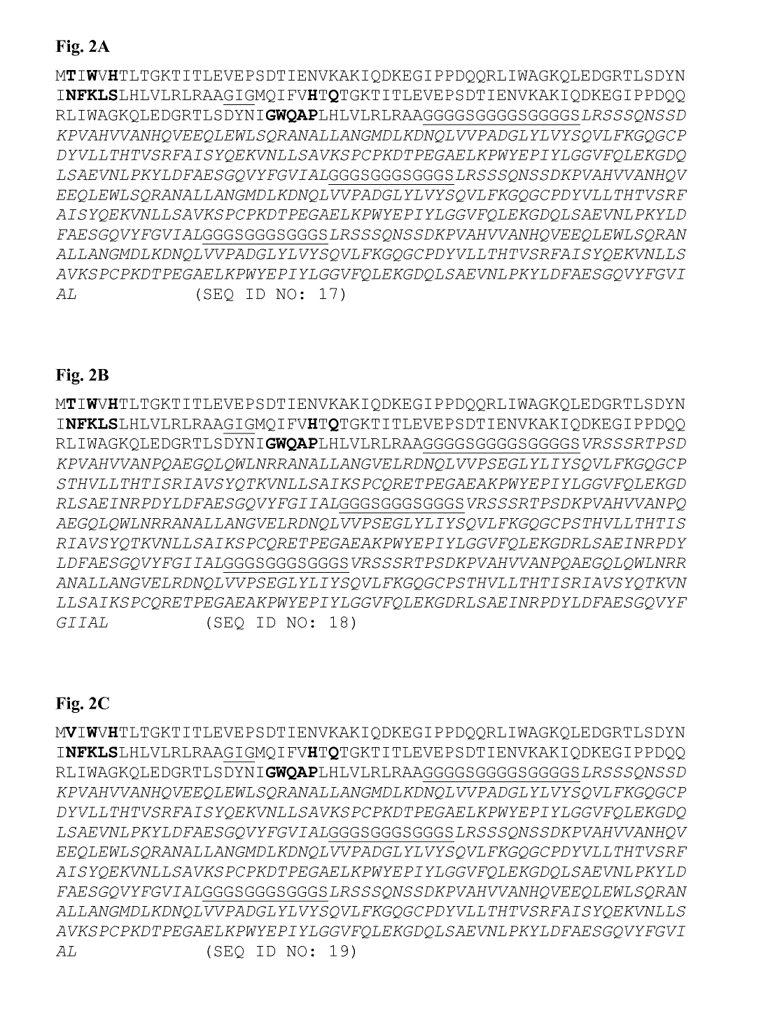

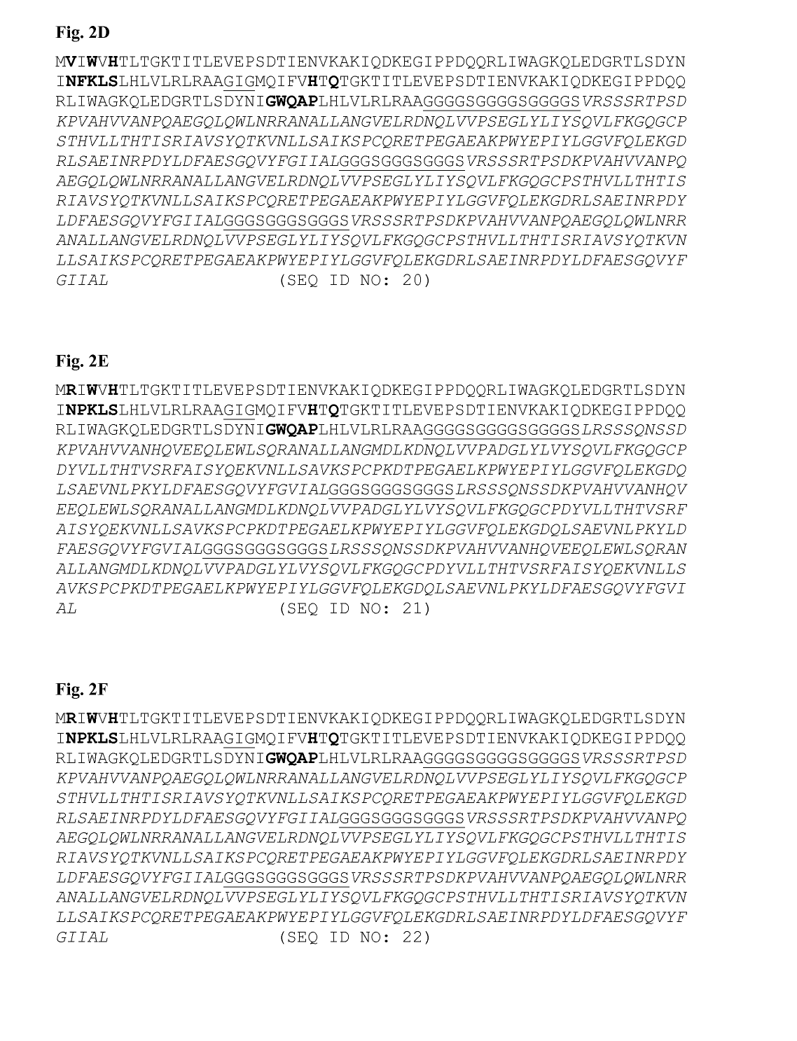

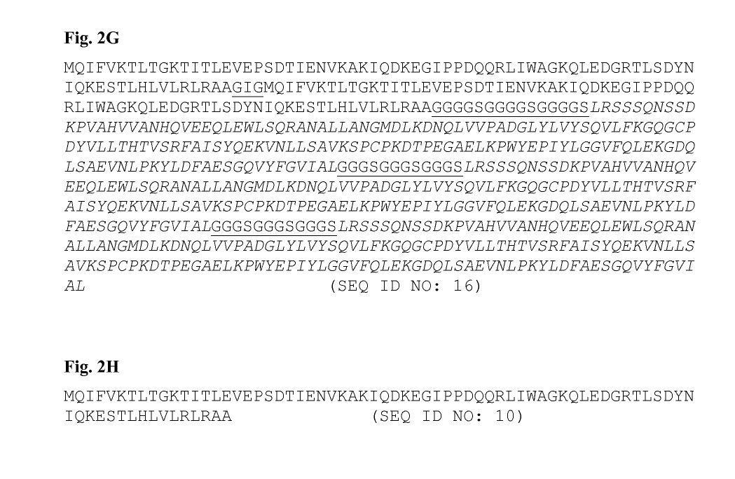

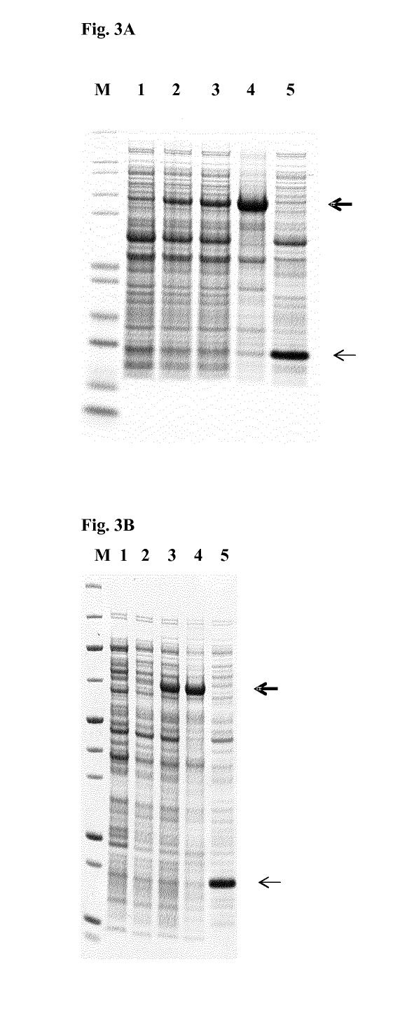

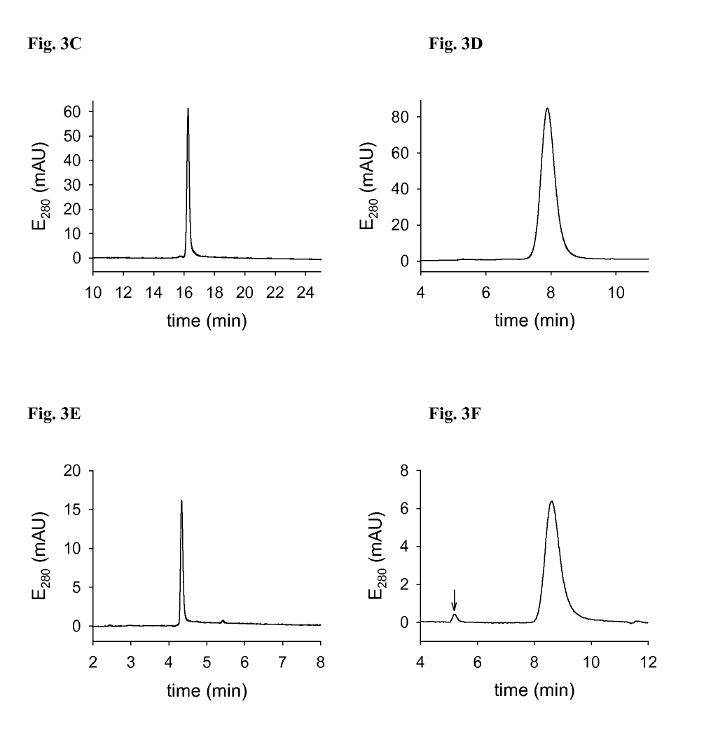

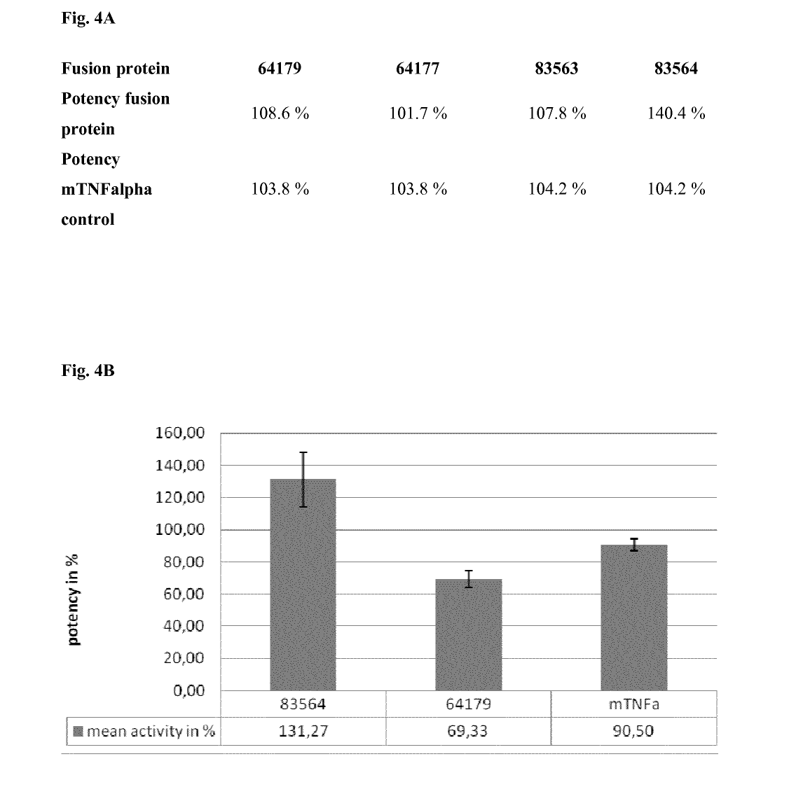

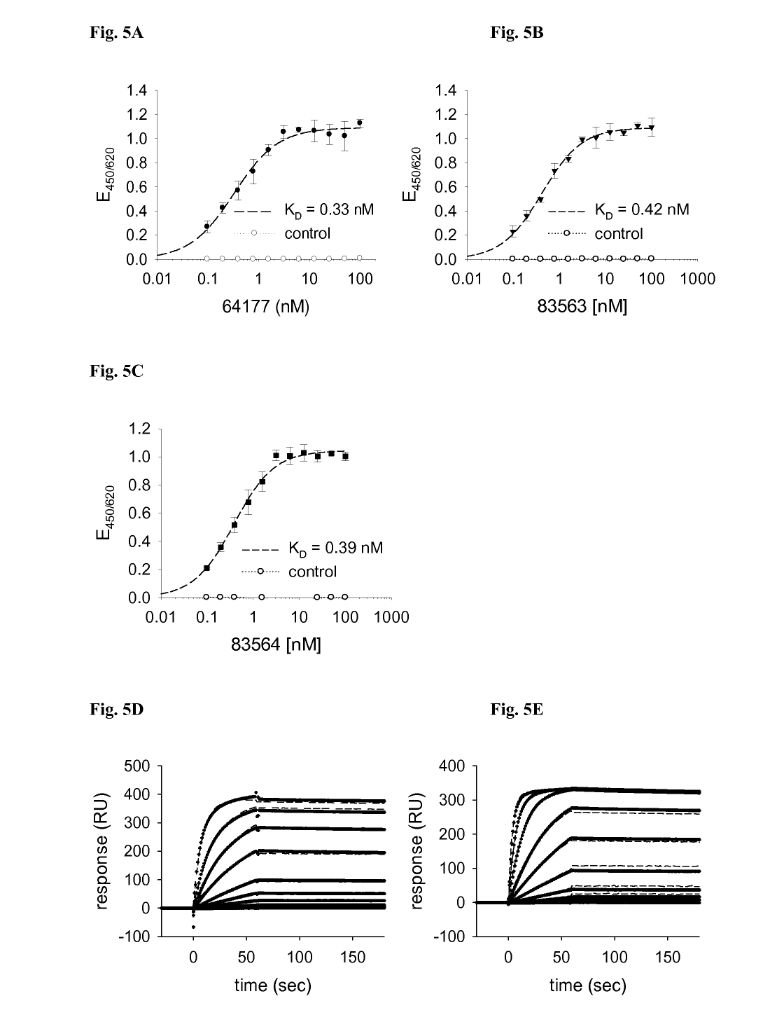

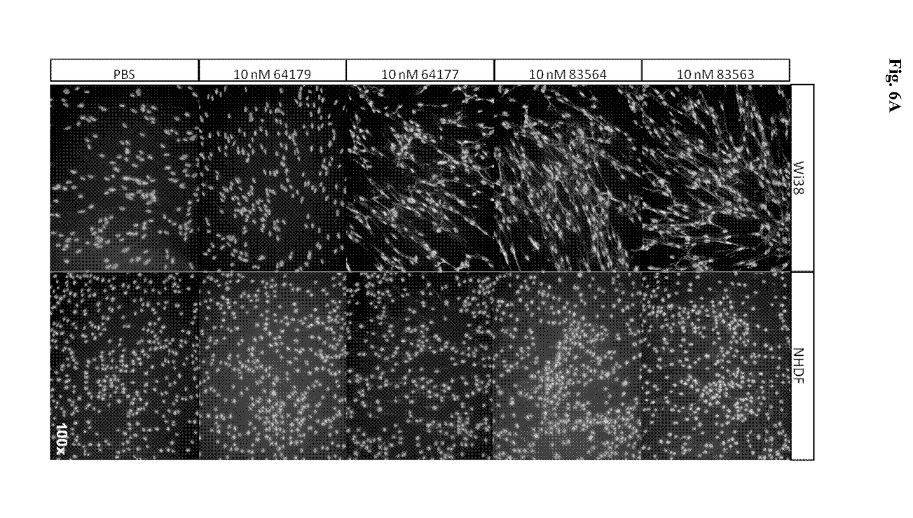

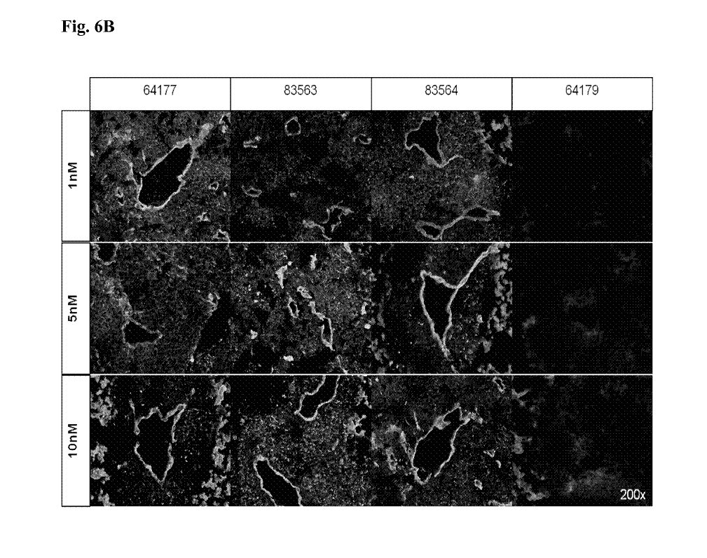

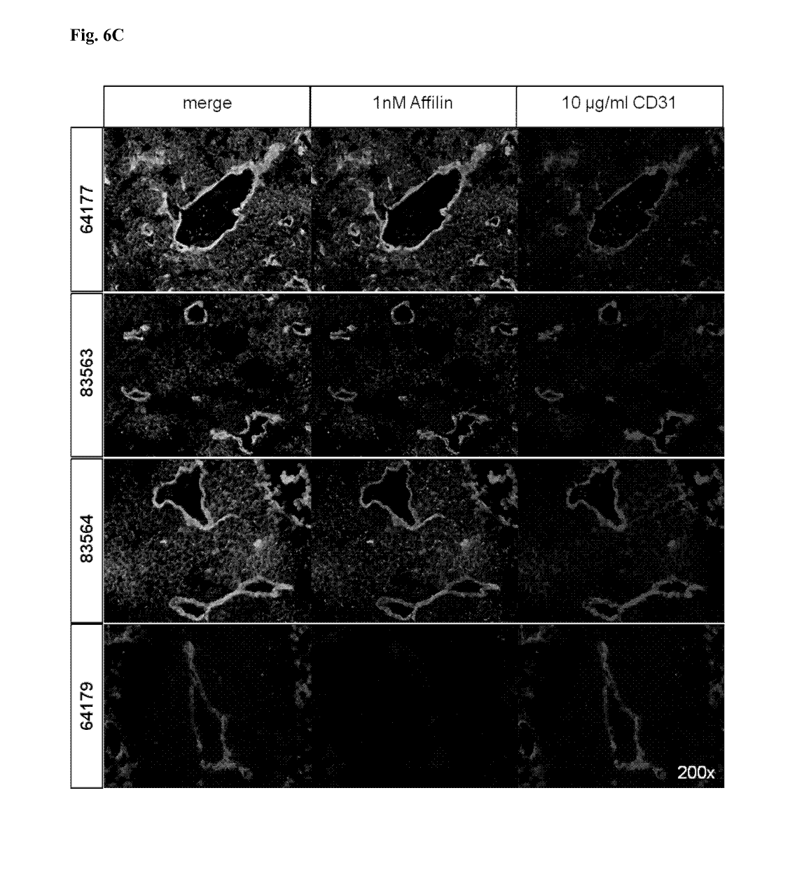

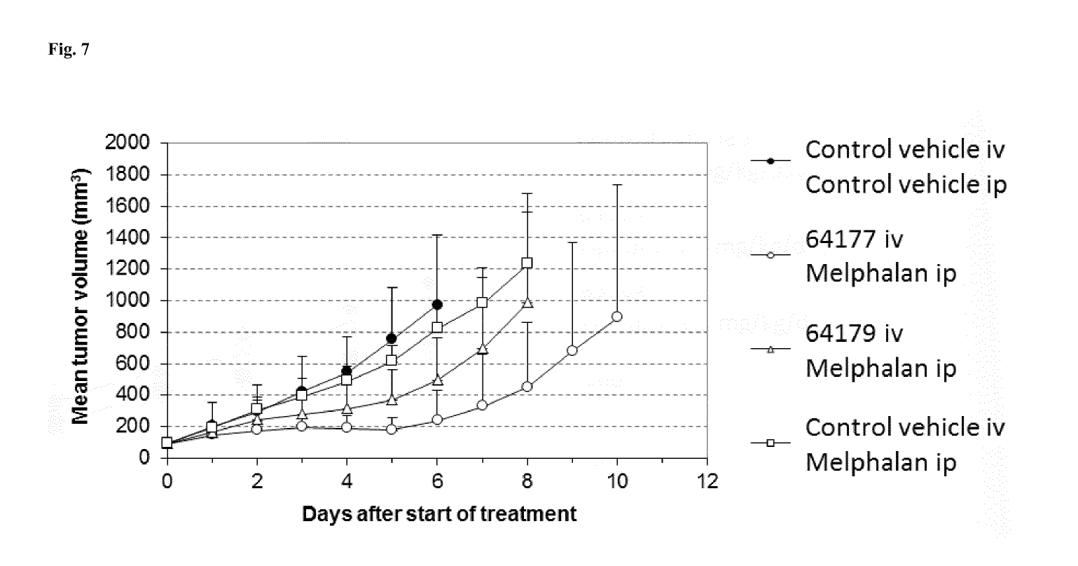

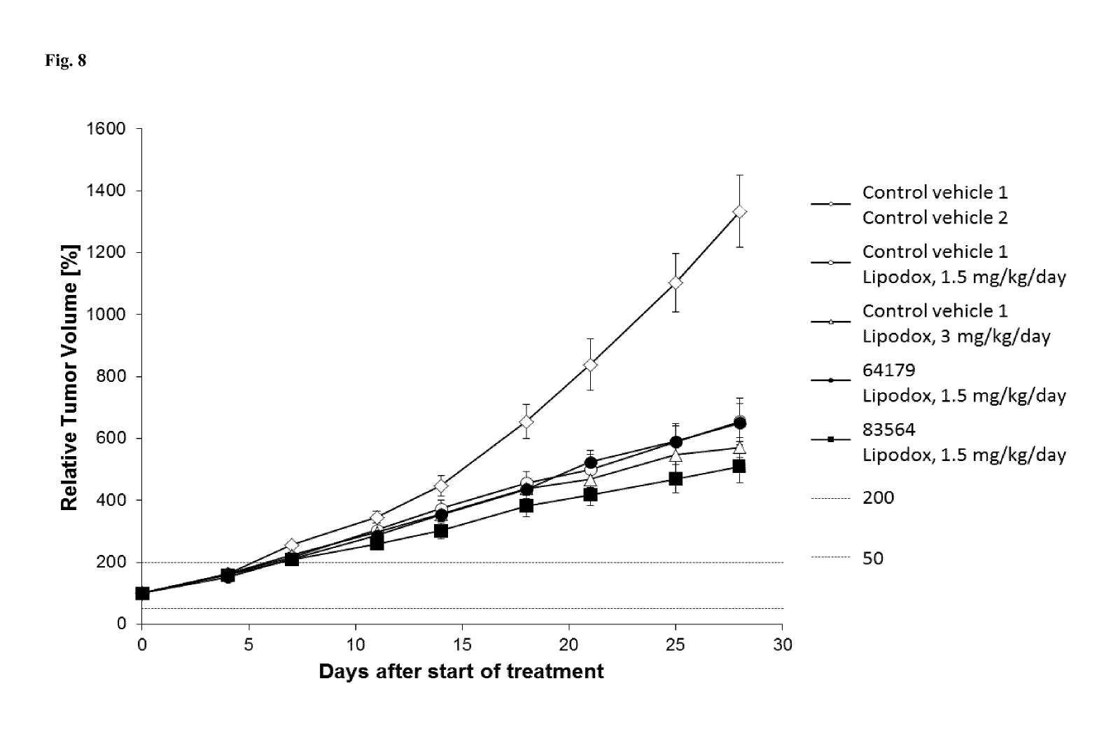

FIG. 2A shows fusion protein 64177 (SEQ ID NO: 17), which comprises anti-ED-B Affilin 54646 and mouse scTNFalpha.

FIG. 2B shows fusion protein 75808 (SEQ ID NO: 18), which comprises anti-ED-B Affilin 54646 and human scTNFalpha.

FIG. 2C shows fusion protein 83563 (SEQ ID NO: 19), which comprises a T2V mutant of anti-ED-B Affilin 54646 and mouse scTNFalpha.

FIG. 2D shows fusion protein h83563 (SEQ ID NO: 20), which comprises a T2V mutant of anti-ED-B Affilin 54646 and human scTNFalpha.

FIG. 2E shows fusion protein 83564 (SEQ ID NO: 21), which comprises a T2R/F63P double mutant of anti-ED-B Affilin 54646 and mouse scTNFalpha.

FIG. 2F shows fusion protein h83564 (SEQ ID NO: 22), which comprises a T2R/F63P double mutant of anti-ED-B Affilin 54646 and human scTNFalpha.

FIG. 2G shows fusion protein 64179 which comprises a non-binding ubiquitin dimer (Ub2) and mouse scTNFalpha (SEQ ID NO: 16).

FIG. 2H shows a monomeric ubiquitin sequence used for modification (SEQ ID NO: 10).

FIG. 3 shows the expression of a fusion protein and analysis of the homogeneity

FIG. 3A shows the expression of fusion protein 64177 (mouse scTNFalpha molecule) by heterologous expression in E. coli cells. Expression was analyzed by SDS-PAGE; lanes were loaded with the following samples. (M) molecular weight marker Mark 12 (Invitrogen); (1) Strain NovaBlue™::pSCIL008b::64177, crude cell extract; (2) Strain NovaBlue™:: pSCIL008b::64177, crude cell extract after 1.5 h of induction; (3) Strain NovaBlue™::pSCIL008b::64177, crude cell extract after 3 h of induction; (4) Strain NovaBlue™::pSCIL008b::64177, insoluble fraction after 3 h of induction; (5) Strain NovaBlue™::pSCIL008b::64177, soluble fraction after 3 h of induction; Bold arrow indicates approximate molecular weight of protein product 64177, light arrow indicates approximate molecular weight of hen egg-white lysozyme used for cell disruption.

FIG. 3B shows the expression of fusion protein 75808 (human scTNFalpha molecule) by heterologous expression in E. coli cells. Expression was analyzed by SDS-PAGE; lanes were loaded with the following samples. (M) Page Ruler™ molecular weight marker (Fermentas); (1) Strain HMS174(DE3)::pSCIL008b::75808 starter culture, crude cell extract; (2) Strain HMS174(DE3)::pSCIL008b:: 75808, crude cell extract before induction; (3) Strain HMS174(DE3)::pSCIL008b::75808, crude cell extract after 4 h of induction; (4) Strain HMS174(DE3)::pSCIL008b:: 75808, insoluble fraction after 4 h of induction; (5) Strain HMS174(DE3)::pSCIL008b:: 75808, soluble fraction after 4 h of induction; Bold arrow indicates approximate molecular weight of protein product 75808, light arrow indicates approximate molecular weight of hen egg-white lysozyme used for cell disruption.

FIG. 3C shows analysis of the homogeneity of a preparation of purified 64177 by reversed-phase HLPC. Reversed-phase HPLC was run of a preparation of purified 64177. Separation was performed on a PLRP-S column (Agilent) in a gradient of 10-80% 2-propanol in 0.1% TFA. Peak integration indicated a purity of >98%.

FIG. 3D shows analysis of the homogeneity of a preparation of purified 64177 by analytical size exclusion chromatography. Separation was performed on a TSKgel G3000SW column (Tosoh Biosciences) in phosphate-buffered saline. Peak integration indicated sample homogeneity of >98%.

FIG. 3E shows analysis of the homogeneity of a preparation of purified 75808 (human scTNFalpha domain) by reversed-phase HPLC. Separation was performed on an Acclaim RSLC 120 C8 column (Dionex) in a gradient of 20-70% acetonitrile in 0.1% TFA. Peak integration indicated a purity of >98%.

FIG. 3F shows analysis of the homogeneity of a preparation of purified 75808 by analytical size exclusion chromatography. Separation was performed on a TSKgel G3000SW column (Tosoh Biosciences) in phosphate-buffered saline. Peak integration indicated sample homogeneity of >98%, with (<2%) protein aggregates (arrow) detectable in the analyzed preparation.

FIG. 4 shows activity of the scTNFalpha domain of the fusion protein

FIG. 4A shows the activity of the scTNFalpha molecule of several TNFalpha fusion proteins. An L929 assay was used as activity assay. As control, mouse TNFalpha was used (referred to as mTNFalpha in the figure). The figure shows that several purified scTNFalpha fusion proteins have the same activity compared to the internal standard mTNFalpha. Therefore, fusion of different heterodimeric ubiquitin variants (Affilin® molecules) to the N-terminus of scTNFalpha does not interfere with the in vitro activity of scTNFalpha.

FIG. 4B shows the TNFalpha activity of fusion protein 83564 in an NFkappaB-RE-assay. Columns shows the proteins analysed: 83564, control protein 64179 and mouse TNFalpha (mTNFa). 83564 shows a high TNF alpha activity.

FIG. 5 shows binding affinity and specificity of the targeting domain of the fusion protein

FIG. 5 A shows the analysis of ED-B binding by the fusion proteins. Binding of fusion protein 64177 to an immobilized human ED-B construct was analyzed by ELISA. The K_{D} determined by least-square fit of the data (dashed line) was 0.33 nM.

FIG. 5 B shows the analysis of ED-B binding by the fusion proteins. Binding of fusion protein 83563 to an immobilized human ED-B construct was analyzed by ELISA. The K_{D} determined by least-square fit of the data (dashed line) was 0.42 nM.

FIG. 5 C shows the analysis of ED-B binding by the fusion proteins. Binding of fusion protein 83564 to an immobilized human ED-B construct was analyzed by ELISA. The K_{D} determined by least-square fit of the data (dashed line) was 0.39 nM.

FIG. 5 D shows a surface plasmon resonance (Biacore™) analysis of the targeting domain (heterodimeric ubiquitin with specificity for ED-B). Biacore analysis of the human ED-B binding activity of the modified ubiquitin moiety is shown for fusion protein 64177 (mouse scTNFalpha moiety). The equilibrium dissociation constant K_{D} determined from a global least-square fit of the data (dashed line) was 0.22 nM, while the determined rate constants were 6.12*10⁵ M⁻¹s⁻¹ for association and 1.36*10⁴s⁻¹ for dissociation, respectively.

FIG. 5 E shows a surface plasmon resonance (Biacore™) analysis of the targeting domain (heterodimeric ubiquitin with specificity for ED-B). Biacore analysis of the human ED-B binding activity of the modified ubiquitin moiety is shown for fusion protein 75808 (human scTNFalpha moiety). The equilibrium dissociation constant K_{D} determined from a global least-square fit of the data (dashed line) was 0.12 nM, while the determined rate constants were 1.33*10⁶ M⁻¹s⁻¹ for association and 1.62*10⁻⁴s⁻¹ for dissociation, respectively.

FIG. 6 shows binding of the fusion proteins to ED-B on vital cells.

FIG. 6A shows staining of native ED-B on vital cells.

Shown is the analysis of the cancer-binding proteins (Affilin) fused to scTNFalpha (64177, 83563 and 83564), the non-targeting domain protein fused to scTNFalpha (64179) and the PBS-control on different cell types. An equimolar dose of 10 nM 83563, 83564 and 64177 shows a staining on EDB-expressing Wi38-cells. NHDF-cells, which are primary normal fibroblast clls expressing low level of EDB-fibronectin, show only a weak matrix staining Binding of the control protein 64179 is not detectable on both cell types. The PBS control shows no unspecific staining of anti-TNFalpha-antibody.

Column 1 shows a strong EDB-staining of 83563, 83564 and 64177 on Wi38 cells. No unspecific staining was observed with the non-binding protein 64179 or the PBS control (row 4, 5). The second column shows the analyses of scTNFalpha-fusion proteins on human normal dermal fibroblast cells. On these low level EDB-expressing cells, only weak binding was observed for the scTNFalpha fusion proteins. The analysis clearly shows that only the targeted fusion proteins binds to vital Wi38 cells with high specificity to ED-B containing extracellular matrix.

FIG. 6 B shows a specific binding of fusion proteins 64177, 83563 and 83564 with different concentrations (1 nM, 5 nM and 10 nM) on F9-tumor tissue. A staining of non-binding fusion protein 64179 was not detected. Variants 64177 (column 1), 83563 (column 2) and 83564 (column 3) showed a strong ED-B binding from 1 to 10 nM. 64179, the scTNFalpha fused non-tumor targeted protein was not detected on F9-tumor slices (column 4).

FIG. 6 C shows the accumulation of 1 nM fusion protein 64177, 83563, 83564 and 64179 closely associated to F9-tumor vessels. Tumor vessels were represented by anti-CD31-staining (single staining, third column). “1 nM Affilin” (second column) refers to a single staining by 1 nM of the dedicated fusion protein. The merged pictures (first column) show the vessel-association of ED-B-binding proteins in F9-tumor slices. All binding proteins 64177, 83563 and 83564 show a strong and predominat vessel-associated staining of F9-tumor slices. 64179, the scTNFalpha fused non-tumor targeted protein shows no accumulation in F9-tumor vessels (row 4).

FIG. 7 shows results of an efficacy study in F9 tumor bearing mice. Tumor growth following combination treatment with a fusion protein of the invention and a cytostatic drug was analyzed. Functionality in terms of tumor growth inhibition by the scTNFalpha moiety of the fusion protein of the invention fused to a non-targeting domain (64179) is compared to the cancer-binding protein fused to scTNFalpha (64177). Further, control groups 1 and 8 (only PBS or PBS plus Melphalan) are shown by closed circles and open squares, respectively. Group 2 (open circle) shows treatment with 64177 plus Melphalan, Group 5 (open triangle) shows treatment with 64179 (Ubi2-scTNF-fusion protein) plus Melphalan. 64177 delays the tumor growth compared to the negative control protein 64179 as well as vehicle and Melphalan control groups by 2 and 4 days, respectively.

FIG. 8 shows results of an efficacy study in breast cancer (MDA-MB 231) tumor bearing mice. Tumor (breast cancer) growth following combination treatment with fusion protein 83564 (control: 64179) and cytostatic drug Lipodox was analyzed. Treatment with cancer-binding protein fused to scTNFalpha (83564) combined with Lipodox shows the lowest relative tumor volume.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

The term “about” when used in connection with a numerical value is meant to encompass numerical values within a range having a lower limit that is 5% smaller than the indicated numerical value and having an upper limit that is 5% larger than the indicated numerical value.

The terms “protein capable of binding” or “binding protein” refer to a protein comprising a binding domain to a target molecule (e.g. a tumor specific protein) as further defined below. Preferably, this term refers in the context of the present invention to binding proteins based on modified ubiquitin molecules. Any such binding protein, for example, based on modified ubiquitin molecules, may comprise additional protein domains that are not binding domains, such as, for example, multimerization moieties, polypeptide tags, polypeptide linkers and/or non-proteinaceous polymer molecules. Some examples of non-proteinaceous polymer molecules are hydroxyethyl starch, polyethylene glycol, polypropylene glycol, or polyoxyalkylene.

Antibodies and fragments thereof are well known to the person skilled in the art. The binding protein of the invention is not an antibody or a fragment thereof. The targeting protein of the invention does not comprise an immunoglobulin fold as present in antibodies.

In the present specification, the terms “ligand” and “target molecule” and “binding partner” are used synonymously and can be exchanged. A ligand is any molecule (e.g. an antigen or a hapten) capable of binding with an affinity as defined herein to a binding protein, which is preferably in the context of the present invention a hetero-multimeric modified ubiquitin protein.

The term “targeting domain” or “targeting moiety” refers to a domain capable of directing the molecules to target expressing cells or cell-associated target structures of interest (e.g., cancer cells, endothelial cells, cancer cell associated matrix or endothelial cell associated matrix).

Preferred “target molecules” when practicing the present invention are proteins and more specifically antigenic epitopes present on proteins. More preferred target molecules are tumor antigens, such as proteins or epitopes that are present on the outside of a tumor cell but that are absent on normal cells of the same tissue-type or which are present in tumor tissue but absent on normal tissue from the same tissue type. A particularly preferred target molecule in the context of the present invention is ED-B of fibronectin.

The term “extra-domain B of fibronectin” or briefly designated as “ED-B” comprises all proteins which show a sequence identity to SEQ ID NO: 2 of at least 70%, optionally at least 75%, further optionally at least 80%, 85%, 90%, 95%, 96% or 97%, or most preferably showing a sequence identity to SEQ ID NO: 2 of 100%, and having the above defined functionality of ED-B (see in particular the above section entitled “Extra-domain B of fibronectin as tumor specific protein”).

The term “fusion protein” relates to a fusion protein comprising a binding or non-binding protein of the invention fused to a functional or an effector component. In one embodiment, the invention relates to a fusion protein comprising a binding protein of the invention as targeting moiety fused to a functional or an effector domain, such as single chain TNFalpha. A fusion protein of the invention may further comprise non-polypeptide components, e.g. non-peptidic linkers, non-peptidic ligands, e.g. for therapeutically or diagnostically relevant radionuclides. It may also comprise small organic or non-amino acid based compounds, e.g. a sugar, oligo- or polysaccharide, fatty acid, etc. In one preferred embodiment of the invention, the heterodimeric modified ubiquitin-based ED-B binding molecule is covalently or non-covalently conjugated to a protein or peptide having therapeutically relevant properties, preferably to scTNFalpha. Methods for covalently and non-covalently attaching a protein of interest to a support are well known in the art, and are thus not described in further detail here.

The term “scTNF” or “scTNFalpha” refers to at least three (e.g. three, six or nine) TNFalpha monomers that are joined by linkers, thereby forming a single chain (sc) TNFalpha (scTNF or scTNFalpha) molecule. Only scTNFalpha molecules with trimeric structure have biological activity. A trimeric structure is required to be able to bind to specific TNF receptors and induce the formation of ligand/receptor complexes. Connecting linkers should be designed in a way so that the TNFalpha monomers are not influenced by the linkers in terms of their biological activity. Linkers between the TNFalpha monomers should allow the binding to specific TNF receptors and allow the formation of ligand/receptor complexes. Connecting linkers between the TNFalpha monomers are preferably peptide linkers of, for example, at least 8 amino acids. A suitable linker is shown in SEQ ID NO: 9.

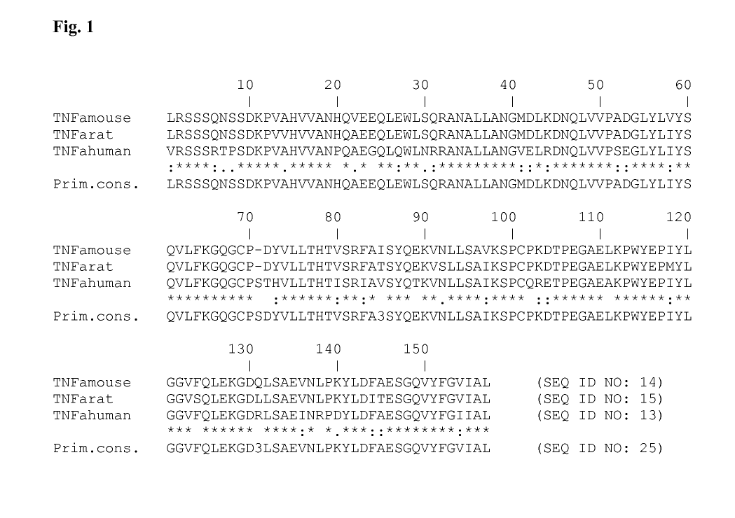

The term “TNFalpha” (or spelling variants thereof such as “TNF-alpha”, “TNFα”, or “TNF alpha”) covers TNFalpha molecules in accordance with SEQ ID NO: 13 (human; uniprot accession number P01375; see: http://www.uniprot.org/uniprot/P01375), SEQ ID NO: 14 (mouse; uniprot accession number P06804; http://www.uniprot.org/uniprot/P06804), SEQ ID NO: 15 (rat) or any other homolog sequences. Human TNFalpha shows 79% sequence identity to mouse TNFalpha. The amino acid sequences shown in SEQ ID NO: 13, 14 and 15 (see FIG. 1) correspond to the sequences of mature native TNFalpha monomers in the respective species after cleavage of the signal peptide. However, as used herein, the term “TNFalpha” also covers amino acid sequences in which a start methionine or leader sequences or tags or other amino acids are added to the amino terminus of SEQ ID NO: 13, SEQ ID NO: 14 or SEQ ID NO: 15.

The term “biologically active TNFalpha” encompasses polypeptides that are sequence variants of SEQ ID NO: 13, SEQ ID NO: 14 or SEQ ID NO: 15 and exhibit the same biological functions as the naturally occurring TNFalpha molecules according to SEQ ID NO: 13, SEQ ID NO: 14 or SEQ ID NO: 15. Such “biologically active TNFalpha” molecules can occur in nature or can be artificially created polypeptides. In the context of the present application, the term “biologically active TNFalpha” especially refers to polypeptides that exhibit at least 90% sequence identity (e.g. at least 91%, at least 92%, at least 93%, at least 94%, at least 95%, at least 96%, at least 97%, at least 98%, or at least 99%) to the amino acid sequence set forth in SEQ ID NO: 13 and that exhibit an apoptotic activity, as does naturally occurring TNFalpha. A sequence variant of SEQ ID NO: 13, SEQ ID NO: 14 or SEQ ID NO: 15 is considered to be a “biologically active TNFalpha” polypeptide for the purposes of the present invention, if said sequence variant exhibits at least 90% of the apoptotic activity of human TNFalpha having the amino acid sequence according to SEQ ID NO: 13. The apoptotic activity can be determined by the L929 cytotoxicity assay described by Flick et al. (1984, J. Immunol. Methods, 68:167-175) and explained in example 3 of this application.

The terms “biologically active single-chain TNFalpha”, “biologically active scTNFalpha” or “biologically active scTNF” refers to at least three (e.g. three, six or nine) monomers of biologically active TNFalpha, wherein the term “biologically active TNFalpha” is defined as above, and wherein these monomers are joined by linkers so that a biologically active single chain (sc) TNFalpha (scTNFalpha or scTNF) molecule is formed.

The term “ubiquitin protein” covers the ubiquitin in accordance with SEQ ID NO: 1 and modifications thereof according to the following definition. Ubiquitin is highly conserved in eukaryotic organisms. For example, in all mammals investigated up to now ubiquitin has the identical amino acid sequence. Particularly preferred are ubiquitin molecules from humans, rodents, pigs, and primates. Additionally, ubiquitin from any other eukaryotic source can be used. For instance ubiquitin of yeast differs only in three amino acids from the sequence of SEQ ID NO: 1. Generally, the ubiquitin proteins covered by said term “ubiquitin protein” show an amino acid identity of at least 70%, preferably at least 75%, more preferably at least 80%, at least 85%, at least 90%, at least 95%, at least 96%, at least 97%, or at least 98% to SEQ ID NO: 1.

The term “a modified ubiquitin protein” refers to modifications of the ubiquitin protein, any one of substitutions, insertions or deletions of amino acids or a combination thereof, while substitutions are the most preferred modifications which may be supplemented by any one of the modifications described above. The number of modifications is strictly limited as said modified monomeric ubiquitin units have an amino acid identity to SEQ ID NO: 1 of one of the group consisting of at least 80%, at least 83%, at least 85%, at least 87% and at least 90%. At the most, the overall number of substitutions in a monomeric unit is, therefore, limited to 15 amino acids corresponding to 80% amino acid identity. The total number of modified amino acids in the hetero-dimeric ubiquitin molecule is 30 amino acids corresponding to 20% amino acid modifications based on the hetero-dimeric protein. The amino acid identity of the dimeric modified ubiquitin protein compared to a dimeric unmodified ubiquitin protein with a basic monomeric sequence of SEQ ID NO: 1 is selected from one of the group consisting of at least 80%, at least 83%, at least 84%, at least 85%, at least 86%, at least 87%, at least 88%, at least 89% and at least 90%.

For determining the extent of sequence identity between two amino acid sequences, for example, the SIM Local similarity program (Xiaoquin Huang and Webb Miller, Advances in Applied Mathematics, vol. 12: 337-357, 1991) or ClustalW can be used (Thompson et al., Nucleic Acids Res., 22(22): 4673-4680, 1994). In particular, the sequence identity percentage between a derivative of ubiquitin and the amino acid sequence of SEQ ID NO: 1 can be determined with either of these programs. Preferably, the default parameters of the SIM Local similarity program or of ClustalW are used, when calculating sequence identity percentages. Preferably, the extent of the sequence identity of the modified protein to SEQ ID NO: 1 is determined relative to the complete sequence of SEQ ID NO: 1.

In the context of the present invention, the extent of sequence identity between a modified sequence and the sequence from which it is derived (also termed: “parent sequence”) is generally calculated with respect to the total length of the unmodified sequence, if not explicitly stated otherwise.

A “dimer” is considered as a protein in this invention which comprises two monomeric ubiquitin proteins. If the dimer comprises two differently modified monomers, it is called a “heteromeric-dimer” or “hetero-dimer”. Thus, the “hetero-dimer” of the invention is considered as a fusion of two differently modified monomeric ubiquitin proteins exhibiting a combined binding property (binding domain or targeting domain) for its specific target molecule (e.g. a tumor antigens such as ED-B or any other antigens).

According to the present invention the two monomeric modified ubiquitin proteins are not linked together after having screened the most potent binding ubiquitin molecules but that already the screening process is performed in the presence of the hetero-dimeric ubiquitins. After having received the sequence information on the most potent binding ubiquitin molecules, these molecules may be obtained by any other method, e.g. by chemical synthesis or by genetic engineering methods, e.g. by linking the two already identified monomeric ubiquitin units together.

According to the invention, the two differently modified ubiquitin monomers which bind to one ligand are to be linked by head-to-tail fusion to each other using e.g. genetic methods. The differently modified fused ubiquitin monomers are only effective if acting together.

A “head to-tail fusion” is to be understood as fusing the C-terminus of the first protein to the N-terminus of the second protein. In a head-to-tail fusion, monomers may be connected directly without any linker, i.e. by a direct peptide bond. Alternatively, the fusion of ubiquitin monomers or of TNFalpha molecules can be performed via linkers.

As used herein, the term “linker” refers to a molecule that joins at least two other molecules either covalently or non-covalently, e.g., through hydrogen bonds, ionic or van der Waals interactions, e.g., a nucleic acid molecule that hybridizes to one complementary sequence at the 5′ end and to another complementary sequence at the 3′ end, thus joining two non-complementary sequences. A “linker” is to be understood in the context of the present application as a moiety that connects a first polypeptide with at least a further polypeptide. The second polypeptide may be the same as the first polypeptide or it may be different.

Preferred herein are “peptide linkers”. This means that the peptide linker is an amino acid sequence that connects a first polypeptide with a second polypeptide. The peptide linker is connected to the first polypeptide and to the second polypeptide by a peptide bond. Typically, a peptide linker has a length of between 1 and 30 amino acids; e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. It is preferred that the amino sequence of the peptide linker is not immunogenic to human beings. For example, a linker comprising at least the amino acid sequence GIG (SEQ ID NO: 3) or comprising at least the amino acid sequence Serine-Glycine (SG), for example SGGGG (or SG₄; SEQ ID NO: 4), SGGGGIG (SEQ ID NO: 5), SGGGGSGGGGIG (SEQ ID NO: 6), SGGGGSGGGG (or (SG₄)₂; SEQ ID NO: 7), GGGGSGGGGSGGGGS (or (G₄S)₃; SEQ ID NO: 8) or GGGSGGGSGGGS (or G₃S)₃; SEQ ID NO: 9), SG(G₄S)₃ (SEQ ID NO: 26), SGGGGGS (or SG₅S; SEQ ID NO: 27), GGGGS (or G₄S; SEQ ID NO: 28), or SSSSGSSSSGSSSSG (or (S₄G)₃; SEQ ID NO: 29), or any other peptide linker can be used in the present invention. Also, other linkers for the fusion of two protein monomers are known in the art and can be used.

For linking the targeting moiety und scTNFalpha, peptide linkers are preferred. Peptide linkers based on amino acids serine and glycine (i.e. SG-linkers) are most preferred. SG-linkers provide the advantage that they show little interaction with the linked proteins, because they do not form internal structures. Accordingly, SG-linkers have little or no effect on the structure and function of the linked proteins. For example, SGGGGSGGGGIG (SEQ ID NO: 6), SGGGGSGGGG (SEQ ID NO: 7), GGGGSGGGGSGGGGS (or (G₄S)₃; SEQ ID NO: 8), GGGSGGGSGGGS (or (G₃S)₃; SEQ ID NO: 9), SGGGGGSGGGGSGGGGS (or SG(G₄S)₃; SEQ ID NO: 26), SGGGGGS (or SG₅S; SEQ ID NO: 27), GGGGS (or G₄S; SEQ ID NO: 28), or SSSSGSSSSGSSSSG (or (S₄G)₃; SEQ ID NO: 29) can be used to link the targeting moiety und scTNF. Preferred for the linkage between the targeting moiety and scTNF are peptide linkers with at least 10 but maximal 30 amino acids. More preferred are linkers with 10 to 20 amino acids. Most preferred are linkers with 12-15 amino acids, preferably GGGGSGGGGSGGGGS (SEQ ID NO: 8) or GGGSGGGSGGGS (SEQ ID NO: 9).

Alternatively, the targeting domain may be connected to a scTNFalpha molecule directly without any linker. This may be suitable for targeted proteins with a more flexible connecting region, e.g. a more flexible C-terminal region in loop structures linked to the N-terminus of scTNFalpha.

In a preferred embodiment of the invention, the targeting domain of the fusion protein is a modified hetero-dimeric ubiquitin protein. In this case, it is preferred that the fusion of the targeting domain to scTNFalpha molecule is performed via linkers, preferably such as the linkers defined by the SEQ ID NOs: 6, 7, 8, 9, 26, or 29. Other linkers for the fusion of two proteins are known to the person skilled in the art.

The modified ubiquitin proteins of the invention are engineered, artificial proteins with novel binding affinities to target molecules. This means that the binding affinity to a target was created de novo by modifying, for example, by substituting certain amino acids in wildtype ubiquitin. After substituting 1-8 amino acids in a ubiquitin monomer and linking two modified ubiquitin monomers, the novel artificial protein—heterodimeric ubiquitin—has novel binding capabilities. The term “substitution” comprises also the chemical modification of amino acids by e.g. substituting or adding chemical groups or residues to the original amino acid.

The substitution of amino acids for the generation of the novel binding domain specific to the target molecules can be performed according to the invention with any desired amino acid, i.e. for the modification to generate the novel binding property to the target molecule it is not mandatory to take care that the amino acids have a particular chemical property or a side chain, respectively, which is similar to that of the amino acids substituted so that any amino acid desired can be used for this purpose.

The step of modification of the selected amino acids is performed according to the invention preferably by mutagenesis on the genetic level by random mutagenesis, i.e. a random substitution of the selected amino acids. Preferably, the modification of ubiquitin is carried out by means of methods of genetic engineering for the alteration of a DNA belonging to the respective protein. Preferably, expression of the ubiquitin protein is then carried out in prokaryotic or eukaryotic organisms.

In preferred embodiments, the amino acid residues that are involved in novel binding capabilities are altered by amino acid substitutions. However, also deletions and/or insertions are allowable modifications. The number of amino acids which may be added is limited to 1, 2, 3, 4, 5, 6, 7, or 8 amino acids in a monomeric ubiquitin subunit, and accordingly 1, 2, 3, 4, 5, 6, 7, 8, 9, 10, 11, 12, 13, 14, 15, 16 amino acids with respect to the dimeric ubiquitin protein. The number of amino acids which may be deleted is limited to 1, 2, 3, 4, 5, 6, 7, or 8 amino acids in a monomeric ubiquitin subunit, and accordingly 1, 2, 3, 4, 5, 6, 7, 8, 9, 10, 11, 12, 13, 14, 15, 16 amino acids with respect to the dimeric ubiquitin protein. In one embodiment, no amino acid insertions are made. In a still further embodiment, no deletions have been performed. In still other embodiments, a number of deletion (e.g. 1, 2, 3, 4, 5, 6, 7, 8, 9, 10, 11, 12, 13, 14, 15, 16 deletions in the hetero-dimeric ubiquitin protein) is combined with a number of insertions (e.g. 1, 2, 3, 4, 5, 6, 7, 8, 9, 10, 11, 12, 13, 14, 15, 16 insertions in the hetero-dimeric ubiquitin protein).

Provided that the modified ubiquitin protein of the present invention comprises additionally to said substitutions specified in the claims and explained herein also deletions and/or additions of one or more amino acids, the amino acid positions given for wild type human ubiquitin (SEQ ID NO: 1) have to be aligned with the modified ubiquitin in order to allot the corresponding proteins to each other. In case of fusion proteins (see below), the numbering (and alignment) of each of the monomeric ubiquitin subunits is done in the same way, i.e. an alignment of, for example, a dimer is started at amino acid position 1 for each respective subunit.

The modified amino acids in a modified monomeric ubiquitin according to the invention used as building unit for a hetero-dimer account for in total up to 20% of amino acids. Considering this, there is a sequence identity to SEQ ID NO: 1 of the modified ubiquitin protein to at least 80%. In further embodiments of the invention, the sequence identity on amino acid level to the amino acid sequence of SEQ ID NO: 1 is at least 83%, at least 85%, at least 87% and furthermore at least 90%, at least 92% or at least 95%. The invention covers also amino acid sequence identities of more than 97% of the modified ubiquitin protein compared to the amino acid sequence of SEQ ID NO: 1.

In a further embodiment of the invention, an ubiquitin is modified in 1-8 amino acids (e.g. 1, 2, 3, 4, 5, 6, 7 or 8 amino acids) in regions 2-8 and/or 62-68, preferably selected from positions 2, 4, 6, 8, 62, 63, 64, 65, 66, and/or 68 of SEQ ID NO: 1 to generate a novel and highly specific binding to a tumor target. In another embodiment, the ubiquitin to be modified in these positions was already pre-modified. For example, further modifications could comprise modifications at amino acids 74 and/or 75 and/or 76 and/or at amino acid 45 to generate better stability or protein-chemical properties but are not involved in binding to a target. A pre-modified ubiquitin that is particularly well-suited for practicing the present invention is shown in SEQ ID NO: 10. More specifically, all embodiments of the present invention that refer to the wild-type ubiquitin sequence according to SEQ ID NO: 1 apply in an analogous manner to the amino acid sequence of SEQ ID NO: 10. This pre-modified ubiquitin contains the amino acid exchanges F45W, G74A and G75A. A modified ubiquitin monomer is obtainable wherein in total up to 6, 7, 8, 9, 10, 11, 12, 13, 14 and a maximum of 15 amino acids of the ubiquitin of SEQ ID NO: 1 or SEQ ID NO: 10 are modified, preferably substituted.

Variations of ubiquitin protein scaffolds differing by amino acid substitutions in the region of the de novo generated artificial binding site from the parental protein and from each other can be generated by a targeted mutagenesis of the respective sequence segments. In this case, amino acids having certain properties such as polarity, charge, solubility, hydrophobicity or hydrophilicity can be replaced or substituted, respectively, by amino acids with respective other properties. Besides substitutions, the terms “mutagenesis” and “modified” and “replaced” comprise also insertions and/or deletions. On the protein level the modifications can also be carried out by chemical alteration of the amino acid side chains according to methods known to those skilled in the art.

A “randomly modified nucleotide or amino acid sequence” is a nucleotide or amino acid sequence which in a number of positions has been subjected to insertion, deletion or substitution by nucleotides or amino acids, the nature of which cannot be predicted. In many cases the random nucleotides (amino acids) or nucleotide (amino acid) sequences inserted will be “completely random” (e. g. as a consequence of randomized synthesis or PCR-mediated mutagenesis). However, the random sequences can also include sequences which have a common functional feature (e. g. reactivity with a ligand of the expression product) or the random sequences can be random in the sense that the ultimate expression product is of completely random sequence with e. g. an even distribution of the different amino acids.

In accordance with the invention, the term “Kd” (or its alternative spelling “K_{D}”) defines the specific binding affinity which is in accordance with the invention in the range of 10⁻⁷-10⁻¹² M. A value of 10⁻⁵ M and below can be considered as a quantifiable binding affinity. Depending on the application a value of 10⁻⁷ M to 10⁻¹¹ M is preferred for e.g. chromatographic applications or 10⁻⁹ to 10⁻¹² M for e.g. diagnostic or therapeutic applications. Further preferred binding affinities are in the range of 10⁻⁷ to 10⁻¹⁰ M, preferably to 10⁻¹¹ M. The methods for determining the binding affinities are known per se and can be selected for instance from the following methods: ELISA, Surface Plasmon Resonance (SPR) based technology (offered for instance by Biacore™), fluorescence spectroscopy, isothermal titration calorimetry (ITC), analytical ultracentrifugation, FACS.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

A “composition” according to the present invention comprises at least two pharmacologically active compounds. These compounds can be administered simultaneously or separately with a time gap of one minute to several days. The compounds can be administered via the same route or differently; e.g. oral administration of one active compound and parenteral administration of another are possible. Also, the active compounds may be formulated in one medicament, e.g. in one infusion solution or as a kit comprising both compounds formulated separately. Also, it is possible that both compounds are present in two or more packages.

A “combination preparation” according to the present invention comprises a fusion protein of the invention together with a pharmaceutically active agent, preferably a cytotoxic or cytostatic agent.

### EMBODIMENTS OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to a fusion protein comprising, essentially consisting of or consisting of the following parts:

(i) a biologically active single chain TNFalpha molecule that comprises at least three TNFalpha monomers joined by linkers;
(ii) a targeting domain that is capable of binding to a target molecule with a specific binding affinity to the target molecule of Kd≦10⁻⁷; and
(iii) optionally a linker between (i) and (ii).

In preferred embodiments of the first aspect, the modified hetero-dimeric ubiquitin protein has a specific binding affinity to the target molecule of Kd≦10⁻⁷, preferably ≦10⁻⁸, more preferably ≦10⁻⁹, even more preferably ≦10⁻¹⁰, and most preferably ≦10⁴¹.

In preferred embodiments of the first aspect, each TNFalpha monomer is mammalian TNFalpha (e.g. mouse TNFalpha, rat TNFalpha or human TNFalpha), preferably human TNFalpha. The amino acid sequences of human, mouse and rat TNFalpha are shown in SEQ ID NOs: 13, 14, and 15, respectively.

In some embodiments of the first aspect, the targeting domain is a non-immunoglobulin, alternative scaffold polypeptide selected from the group consisting of Affilin® molecules, Anticalins, designed ankyrin repeat proteins (DARPin), Affibody® molecules, Fynomers, nanobodies, maxybodies, avimers, and others (for review see Binz H. K. et al. (2005) Nat. Biotechnol. 23(10):1257-1268).

In particularly preferred embodiments of the first aspect, the targeting domain consists of a modified dimeric ubiquitin protein with high specificity for a pre-defined target (Affilin®). In some embodiments of the present invention, the two ubiquitin parts of said modified dimeric ubiquitin protein have the identical amino acid sequence, i.e. the targeting domain consists of a modified homodimeric ubiquitin protein. However, in most embodiments of the present invention the two ubiquitin parts have been differently modified so that the targeting domain consists of a modified heterodimeric ubiquitin protein. In further preferred embodiments of the first aspect, the modified dimeric ubiquitin protein comprises two monomeric ubiquitin units linked together in a head-to-tail arrangement. In some embodiments, these two monomeric ubiquitin units are directly linked, i.e. without a linker. Alternatively, these two monomeric ubiquitin units may be linked by a linker sequence, e.g. by the linker sequences shown in SEQ ID NOs: 3, 4, 5, 6, 7, 8, 9, 26, 27, 28, or 29 or by the dipeptide linker SG. A particularly well-suited linker sequence is GIG (SEQ ID NO: 3).

### Regions to be Modified in Ubiquitin

The regions for modification can be basically selected as to whether they can be accessible for the target molecule as binding partner and whether the overall structure of the protein will presumably show tolerance to a modification. Particularly preferred are two regions for modification of amino acids within the ubiquitin molecule. The first preferred region is between amino acids 2-8, the second region is between amino acids 62-68.

In preferred embodiments of the first aspect, each monomeric ubiquitin unit in said modified hetero-dimeric ubiquitin protein is modified independently from the modifications in the other monomeric ubiquitin unit by substitutions of 1-8 amino acids selected from positions 2, 4, 6, 8, 62, 63, 64, 65, 66, or 68 of SEQ ID NO: 1 or SEQ ID NO: 10. Other positions might be suitable for substitution as well. Important is that after modification of ubiquitin monomers, that a high specific binding to a cancer target is generated and that the structure of ubiquitin is maintained.

In preferred embodiments of the first aspect, each modified monomeric ubiquitin unit has an amino acid sequence identity of at least 80% (e.g. at least 83%, at least 85%, at least 87%, at least 90%, at least 92%, at least 95%, or at least 97%) to the amino acid sequence defined by SEQ ID NO: 1 or SEQ ID NO: 10.

In preferred embodiments of the first aspect, the substitutions in the monomeric ubiquitin units comprise substitutions in amino acid region 2-8 and in amino acids in region 62-66. In particular, both first monomeric units, substitutions at least selected from amino acid positions 6, 62, 63, 64, 65, 66 and optionally further modifications, preferably substitutions of other amino acids, are preferred.

In preferred embodiments of the first aspect, the target molecule is a tumor antigen. In particularly preferred embodiments, the target molecule is the extradomain B (ED-B) of fibronectin. However, other tumor specific proteins could be used as targets.

Thus, in one embodiment the targeting domain is a genetically fused hetero-dimer of said ubiquitin monomers having different amino acids substitutions in the first ubiquitin monomer and in the second ubiquitin monomer, preferably as shown in Table 1.

TABLE 1
Substitutions in ubiquitin monomers relevant for specific targeting to a tumor

| | ubiquitin monomer 1 | | | | | | | | ubiquitin monomer 2 | | | | | | |
|---|---|---|---|---|---|---|---|---|---|---|---|---|---|---|---|
| variant | 2 | 4 | 6 | 62 | 63 | 64 | 65 | 66 | 6 | 8 | 62 | 63 | 64 | 65 | 66 |
| 54646 (SEQ ID NO: 12 | T | W | H | N | F | K | L | S | H | Q | G | W | Q | A | P |
| mutein T2V of 54646 | V | W | H | N | F | K | L | S | H | Q | G | W | Q | A | P |
| (SEQ ID NO: 23) | | | | | | | | | | | | | | | |
| mutein T2R/F63P of 54646 | R | W | H | N | P | K | L | S | H | Q | G | W | Q | A | P |
| (SEQ ID NO: 24 | | | | | | | | | | | | | | | |

Additionally, the following substitutions are preferred:
in the first ubiquitin monomer substitutions
   Glutamine (Q) to Threonine (T) or Valine (V) or Arginine (R) in position 2,
   Phenylalanine (F) to Tryptophan (W) in position 4,
   Lysine (K) to Histidine (H) in position 6,
   Glutamine (Q) to Asparagine (N) in position 62,
   Lysine (K) to Phenylalanine (F) or Proline (P) in Position 63,
   Glutamic acid (E) to Lysine (K) in position 64,
   Serine (S) to Leucine (L) in position 65, and/or
   Threonine (T) to Serine (S) in position 66;
in the second ubiquitin monomer, the substitutions
   Lysine (K) to Histidine (H) in position 6,
   Leucine (L) to Glutamine (Q) in position 8,
   Glutamine (Q) to Glycine (G) in position 62,
   Lysine (K) to an amino acid with an aromatic side chain, preferably
   Tryptophan (W) or Tyrosine (Y), in position 63,
   Glutamic acid (E) to an amino acid containing an —NH₂ group or an —NH— group in the side chain, preferably Glutamine (Q) or Histidine (H), in position 64,
   Serine (S) to an amino acid that has no voluminous side chain, preferably Alanine (A) or Aspartic acid (D), in position 65, and/or
   Threonine (T) to Proline (P) in position 66.

The alternative substitutions in the second monomer can be combined with each other without any limitations provided that the resulting modified ubiquitin hetero-dimers (Affilin®) show a specific binding affinity to said extradomain B (ED-B) of fibronectin of Kd≦10⁻⁷ M and provided that the structural stability of the ubiquitin protein is not destroyed or hampered. Other amino acid substitutions could be possible, provided that a specific binding affinity to ED-B of Kd≦10⁻⁷ M is achieved.

Fusion Proteins of the Invention

In preferred embodiments of the first aspect, the single chain TNFalpha molecule (which is formed from at least three TNFalpha monomers joined by linkers) is positioned C-terminally to the targeting domain (e.g. a modified hetero-dimeric ubiquitin protein). Alternatively, the single chain TNFalpha molecule (which is formed from at least three TNFalpha monomers joined by linkers) is positioned N-terminally to the targeting domain (e.g. a modified hetero-dimeric ubiquitin protein).

In some embodiments of the first aspect, the linker is absent and the single chain TNFalpha molecule (which is formed from at least three single chain TNFalpha monomers joined by linkers) and the targeting domain are directly fused to each other.

In more preferred embodiments of the first aspect, the linker is present and three single chain TNFalpha molecules and the targeting domain (e.g. the modified hetero-dimeric ubiquitin protein) are connected via a linker, preferably a peptide linker.

In most preferred embodiments, the order of the parts of the fusion protein from the N-terminus to the C-terminus is as follows: modified hetero-dimeric ubiquitin protein—linker 3—TNFalpha monomer—linker 1—TNFalpha monomer—linker 2—TNFalpha monomer, wherein linker 1, linker 2, and linker 3 may all be different or two of linker 1, linker 2, and linker 3 may be the same or all three of linker 1, linker 2, and linker 3 may be the same. It is preferred that linker 3 is a peptide linker of at least 10 amino acids.

Alternatively, the order of the parts of the fusion protein from the N-terminus to the C-terminus is as follows: TNFalpha monomer—linker 1—TNFalpha monomer—linker 2—TNFalpha monomer—linker 3—modified hetero-dimeric ubiquitin protein, wherein linker 1, linker 2, and linker 3 may all be different or two of linker 1, linker 2, and linker 3 may be the same or all three of linker 1, linker 2, and linker 3 may be the same.

Linker 1, linker 2, and linker 3 may be selected independently from each other from the group of linkers consisting of SG, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 26, SEQ ID NO: 27, SEQ ID NO: 28, and SEQ ID NO: 29. However, other peptide linkers known to the person skilled in the art may also be employed.

It is preferred that linker 1 and linker 2 are the same peptide linkers.

It is particularly preferred that linker 3 between the targeting moiety and the scTNF moiety of the fusion protein is a peptide linker, especially an SG-linker, with at least 10 but maximal 30 amino acids. It is more preferred that linker 3 is a peptide linker with 10 to 20 amino acids. It is most preferred that linker 3 is a peptide linker with 12-15 amino acids, preferably the peptide linker as set forth in SEQ ID NO: 8 or in SEQ ID NO: 9.

It is particularly preferred that linker 1 and linker 2 are peptide linkers, especially an SG-linker, with at least 10 but maximal 30 amino acids. It is more preferred that linker 1 and linker 2 are peptide linkers with 10 to 20 amino acids. It is most preferred that linkers 1 and 2 are peptide linkers with 12-15 amino acids, preferably the peptide linker as set forth in SEQ ID NO: 8 or in SEQ ID NO: 9.

The purpose of employing a comparably long peptide linker (i.e. a linker with 10 or more amino acids) as linker 1, 2 and/or 3 lies in the fact that such a long linker provides sufficient flexibility so that the interaction of the connected TNFalpha monomers with each other is not impaired.

In a preferred embodiment, the following linkers are used:

Ubiquitin monomer 1—peptide linker or no linker—ubiquitin monomer 2—Peptide linker of at least 10 amino acids (e.g. SEQ ID NO: 8 or SEQ ID NO: 9)—TNF—Peptide linker of at least 10 amino acids (e.g. SEQ ID NO: 8 or SEQ ID NO: 9)—TNF—Peptide linker of at least 10 amino acids (e.g. SEQ ID NO: 8 or SEQ ID NO: 9)—TNF.

In further preferred embodiments of the first aspect, the targeting moiety is a modified hetero-dimeric ubiquitin protein (Affilin) comprising, essentially consisting of or consisting of an amino acid sequence selected from the group consisting of:

SEQ ID NO: 12, SEQ ID NO: 23, SEQ ID NO: 24, and an amino acid sequence that exhibits at least 90% sequence identity to one or more of the amino acid sequences according to SEQ ID NOs: 12, 23, or 24, provided that said modified hetero-dimeric ubiquitin protein exhibits a specific binding affinity to the extradomain B (ED-B) of fibronectin of Kd≦10⁻⁷.

In further preferred embodiments of the first aspect, the fusion protein comprises, essentially consists of or consists of an amino acid sequence selected from the group consisting of: SEQ ID NO: 17, SEQ ID NO: 18, SEQ ID NO: 19, SEQ ID NO: 20, SEQ ID NO: 21, and SEQ ID NO: 22, and an amino acid sequence that exhibits at least 90% sequence identity to one or more of the amino acid sequences according to SEQ ID NOs: 17 to 22, provided that said modified hetero-dimeric ubiquitin protein exhibits a specific binding affinity to the extradomain B (ED-B) of fibronectin of Kd≦10⁻⁷.

In some embodiments of the first aspect, a fusion protein of the invention may comprise non-polypeptide components, e.g. non-peptidic linkers, non-peptidic ligands, e.g. for therapeutically relevant radionuclides. It may also comprise small organic or non-amino acid based compounds, e.g. a sugar, oligo- or polysaccharide, fatty acid, etc.

Fusion proteins comprising a hetero-dimeric ubiquitin targeting moiety (Affilin) and scTNFalpha are preferably obtained by peptidic or proteinogenic conjugations, i.e. by genetic fusions. Furthermore, fusion proteins comprising a hetero-dimeric ubiquitin targeting moiety and scTNFalpha may be prepared by other methods for covalently or non-covalently attaching a protein moiety to another protein moiety, which are known in the art, and are thus not described in further detail here.

In a further embodiment of the invention the fusion protein according to the invention may contain artificial amino acids.

A further embodiment relates to fusion proteins according to the invention, further comprising functional components selected from proteins, peptides, polymers (e.g. polyethylene glycol), low molecular weight compounds, sugars and others, as described in WO 2006/040129, which is incorporated herein by reference.

In a second aspect the present invention is directed to the fusion protein according to the first aspect for use in medicine. In other words, the present invention relates to the fusion protein of the first aspect for use in a monotherapy.

In a third aspect the present invention is directed to the fusion protein according to the first aspect for use in the treatment of cancer. In other words, the present invention relates to the fusion protein of the first aspect for use as monotherapy in the treatment of cancer.

The third aspect of the present invention can alternatively be worded as follows: In a third aspect the present invention is directed to a method for treating cancer, comprising the step: administering a therapeutic amount of the fusion protein according to the first aspect to a subject in need thereof.

In preferred embodiments of the third aspect, the cancer is selected from the group consisting of breast cancer, colorectal cancer, hepatocellular cancer, follicular lymphoma, melanoma, osteosacroma, pancreas, prostate, lung cancer, renal cell cancer, leukaemia, multiple myeloma, cutaneous T cell lymphoma, carcinoid tumor, glioblastoma multiforme (brain), mesothelioma, squamous cell carcinoma, cell carcinoma, and Hodgkin lymphoma.

In a fourth aspect the present invention is directed to a polynucleotide encoding the fusion protein as defined in the first aspect. In a further embodiment of the fourth aspect, the polynucleotide is for use in medicine, e.g. for use in the treatment of cancer.

One embodiment of the present invention pertains to a method for treating cancer, comprising the step: administering a therapeutic amount of the polynucleotide according to the fourth aspect to a subject in need thereof. The cancer to be treated in accordance with the fourth aspect is preferably selected from the same list of cancers as defined above for the third aspect.

In some embodiments of the fourth aspect, polynucleotides are operatively linked to expression control sequences allowing expression of the fusion proteins of the invention in prokaryotic and/or eukaryotic host cells. Such expression control sequences include but are not limited to inducible and non-inducible, constitutive, cell cycle regulated, metabolically regulated promoters, enhancers, operators, silencers, repressors and other elements that are known to those skilled in the art and that drive or otherwise regulate gene expression. Such regulatory elements include but are not limited to regulatory elements directing constitutive expression like, for example, promoters transcribed by RNA polymerase III like, e.g. promoters for the snRNA U6 or scRNA 7SK gene, the cytomegalovirus hCMV immediate early gene, the early or late promoters of SV40 adenovirus, viral promoter and activator sequences derived from, e.g. NBV, HCV, HSV, HPV, EBV, HTLV, MMTV or HIV; which allow inducible expression like, for example, CUP-I promoter, the tet-repressor as employed, for example, in the tet-on or tet-off systems, the lac system, the trp, system; regulatory elements directing tissue specific expression, regulatory elements directing cell cycle specific expression like, for example, cdc2, cdc25C or cyclin A; or the TAC system, the TRC system, the major operator and promoter regions of phage A, the control regions of fd coat protein, the promoter for 3-phosphoglycerate kinase, the promoters of acid phosphatase, and the promoters of the yeast α- or a-mating factors.

In a fifth aspect the present invention is directed to a vector comprising the polynucleotide of the fourth aspect. In a further embodiment of the fifth aspect, the vector is for use in medicine, e.g. for use in the treatment of cancer.

One embodiment of the present invention pertains to a method for treating cancer, comprising the step: administering a therapeutic amount of the vector according to the fifth aspect to a subject in need thereof. The cancer to be treated in accordance with the fifth aspect is preferably selected from the same list of cancers as defined above for the third aspect.

Vectors suitable for use in the present invention comprises without limitation plasmids, phagemids, phages, cosmids, artificial mammalian chromosomes, knock-out or knock-in constructs, viruses, in particular adenoviruses, vaccinia viruses, attenuated vaccinia viruses, canary pox viruses, lentivirus, herpes viruses, in particular Herpes simplex virus (HSV-I), baculovirus, retrovirus, adeno-associated-virus (AAV), rhinovirus, human immune deficiency virus (HIV), filovirus and engineered versions thereof, virosomes, “naked” DNA liposomes, and nucleic acid coated particles, in particular gold spheres.

In order to express cDNAs encoding the fusion proteins, one typically subclones cDNA into an expression vector that contains a strong promoter to direct transcription, a transcription/translation terminator, and a ribosome-binding site for translational initiation. Suitable bacterial promoters are well known in the art, e.g., E. coli, Bacillus sp., and Salmonella, and kits for such expression systems are commercially available. Similarly eukaryotic expression systems for mammalian cells, yeast, and insect cells are well known in the art and are also commercially available. The eukaryotic expression vector may be, for example an adenoviral vector, an adeno-associated vector, or a retroviral vector.

In a sixth aspect the present invention is directed to a host cell comprising: a fusion protein as defined in the first aspect; a polynucleotide as defined in the fourth aspect; or a vector as defined in the fifth aspect. In a further embodiment of the sixth aspect, the host cell is for use in medicine, e.g. for use in the treatment of cancer.

One embodiment of the present invention pertains to a method for treating cancer, comprising the step: administering a therapeutic amount of the host cell according to the sixth aspect to a subject in need thereof. The cancer to be treated in accordance with the sixth aspect is preferably selected from the same list of cancers as defined above for the third aspect.

A host cell according to the sixth aspect includes but is not limited to prokaryotic cells such as bacteria (for example, E. coli or B. subtilis), which can be transformed with, for example, recombinant bacteriophage DNA, plasmid DNA, or cosmid DNA expression vectors containing the polynucleotide molecules of the invention; simple eukaryotic cells like yeast (for example, Saccharomyces and Pichia), which can be transformed with, for example, recombinant yeast expression vectors containing the polynucleotide molecule of the invention; insect cell systems like, for example, Sf9 or Hi5 cells, which can be infected with, for example, recombinant virus expression vectors (for example, baculovirus) containing the polynucleotide molecules; amphibian cells, e.g. Xenopus oocytes, which can be injected with, for example, plasmids; plant cell systems, which can be infected with, for example, recombinant virus expression vectors (for example, cauliflower mosaic virus (CaMV) or tobacco mosaic virus (TMV)) or transformed with recombinant plasmid expression vectors (for example, Ti plasmid) containing polynucleotide sequences of the invention; or mammalian cell systems (for example, COS, CHO, BHK, HEK293, VERO, HeLa, MDCK, Wi38, and NIH 3T3 cells), which can be transformed with recombinant expression constructs containing, for example, promoters derived, for example, from the genome of mammalian cells (for example, the metallothionein promoter) from mammalian viruses (for example, the adenovirus late promoter and the vaccinia virus 7.5K promoter) or from bacterial cells (for example, the tet-repressor binding is employed in the tet-on and tet-off systems). Also useful as host cells are primary or secondary cells obtained directly from a mammal and transfected with a plasmid vector or infected with a viral vector. Depending on the host cell and the respective vector used to introduce the polynucleotide of the invention the polynucleotide can integrate, for example, into the chromosome or the mitochondrial DNA or can be maintained extrachromosomally like, for example, episomally or can be only transiently comprised in the cells.

In a seventh aspect the present invention relates to the fusion protein according to the first aspect for use in medicine, wherein the fusion protein is for administration in combination with a chemotherapeutic agent. In one embodiment of the seventh aspect, the chemotherapeutic agent is selected from the group of alkylating agents, platinum analogs, antibiotics, taxanes, intercalating agents (e.g. anthracyclines), anti-metabolites, mitosis inhibitors and topoisomerase inhibitors, consisting of, for example, but not limited to, Melphalan, Lipodox, doxorubicin, cyclophosphamide, dactinomycin, fluorodesoxyuracil, cisplatin, paclitaxel, gemcitabine, Docetaxel, Doxil, Myocet, Abraxane, Folfox/Folfiri, Carboplatinum, Pemetrexed, Irinotecan, Capecitabine, Vinorelbine, Epirubicin, Mitoxantron, or radiopharmaceuticals and others.

In an eighth aspect the present invention relates to the fusion protein according to the first aspect for use in the treatment of cancer, wherein the fusion protein is for administration in combination with a chemotherapeutic agent.

The eighth aspect of the present invention can alternatively be worded as follows: In an eighth aspect the present invention is directed to a method for treating cancer, comprising the steps: administering a therapeutic amount of the fusion protein according to the first aspect to a subject in need thereof; and administering a therapeutic amount of a chemotherapeutic agent to said subject.

In one embodiment of the eighth aspect, the chemotherapeutic agent is selected from the group consisting of alkylating agents, platinum analogs, antibiotics, taxanes, intercalating agents (e.g. anthracyclines), anti-metabolites, mitosis inhibitors and topoisomerase inhibitors, consisting of, for example, but not limited to, Melphalan, Lipodox, doxorubicin, cyclophosphamide, dactinomycin, fluorodesoxyuracil, cisplatin, paclitaxel, gemcitabine, Docetaxel, Doxil, Myocet, Abraxane, Folfox/Folfiri, Carboplatinum, Pemetrexed, Irinotecan, Capecitabine, Vinorelbine, Epirubicin, Mitoxantron, or radiopharmaceuticals and others.

In a ninth aspect the present invention is directed to a pharmaceutical composition comprising: a fusion protein as defined in the first aspect; a polynucleotide as defined in the fourth aspect; a vector as defined in the fifth aspect; or a host cell as defined in the sixth aspect; and further comprising a pharmaceutically acceptable carrier. In a particularly preferred embodiment of the ninth aspect, the pharmaceutical composition further comprises one or more chemotherapeutic agents, such as alkylating agents, platinum analogs, antibiotics, taxanes, intercalating agents (e.g. anthracyclines), anti-metabolites, mitosis inhibitors and topoisomerase inhibitors, consisting of, for example, but not limited to, Melphalan, Lipodox, doxorubicin, cyclophosphamide, dactinomycin, fluorodesoxyuracil, cisplatin, paclitaxel, gemcitabine, Docetaxel, Doxil, Myocet, Abraxane, Folfox/Folfiri, Carboplatinum, Pemetrexed, Irinotecan, Capecitabine, Vinorelbine, Epirubicin, Mitoxantron, or radiopharmaceuticals and others. The pharmaceutical composition can be made alone as monotherapy or in the form of a combined preparation or in the form of a kit of parts.

Fusion proteins according to the invention may be prepared by any of the many conventional and well known techniques such as plain organic synthetic strategies, solid phase-assisted synthesis techniques or by commercially available automated synthesizers. On the other hand, they may also be prepared by conventional recombinant techniques alone or in combination with conventional synthetic techniques.

In a tenth aspect the present invention is directed to a method for the preparation of a fusion protein as defined in the first aspect, said method comprising the following steps:
(a) preparing a nucleic acid encoding a fusion protein as defined in the first aspect;
(b) introducing said nucleic acid into an expression vector;
(c) introducing said expression vector into a host cell;
(d) cultivating the host cell;
(e) subjecting the host cell to culturing conditions under which a fusion protein is expressed from said vector, thereby producing a fusion protein as defined in the first aspect;
(f) optionally isolating the fusion protein produced in step (e).

In one embodiment of the tenth aspect, the fusion protein produced in step (e) is in the form of inclusion bodies. In a further preferred embodiment of the tenth aspect, the method further comprises the steps: isolating the inclusion bodies; solubilizing said inclusion bodies, thereby obtaining soluble fusion proteins; and further purifying the soluble fusion proteins obtained in the preceding step by at least two chromatographic steps. Suitable chromatographic steps include without limitation size-exclusion chromatography, anion exchange chromatography and cation exchange chromatography.

In an eleventh aspect the present invention is directed to a method for generation of a fusion protein as defined in the first aspect, said method comprising the following steps:

(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified;

(b) providing a target molecule as potential ligand;

(c) contacting said population of differently modified proteins with said target molecule;

(d) identifying a modified dimeric ubiquitin protein by a screening process, wherein said modified dimeric ubiquitin protein binds to said target molecule with a specific binding affinity of Kd≦10⁻⁷ M (preferably ≦10⁻⁸, more preferably ≦10⁻⁹, even more ≦10⁻¹⁰, and most preferably ≦10⁻¹¹);

(e) isolating said modified dimeric ubiquitin protein with said binding affinity;

(f) identifying a polynucleotide sequence encoding the modified dimeric ubiquitin protein of step (e);

(g) preparing a nucleic acid molecule comprising in frame:

(1) the polynucleotide sequence of step (f);

(2) a polynucleotide sequence encoding a biologically active single chain TNFalpha molecule;

(3) optionally a polynucleotide sequence encoding a peptide linker, wherein this polynucleotide sequence encoding a peptide linker is positioned between the polynucleotide sequence according to (1) and the polynucleotide sequence according to (2);

(h) introducing the nucleic acid molecule prepared in step (g) into an expression vector;

(i) introducing said expression vector into a host cell;

(j) subjecting the host cell to culturing conditions under which a protein is expressed from said vector, thereby producing a fusion protein comprising the modified dimeric ubiquitin protein identified in step (d), a biologically active single chain TNFalpha molecule; and optionally a peptide linker; and

(k) optionally isolating the fusion protein produced in step (j).

In preferred embodiments of the eleventh aspect, each monomer of said dimeric protein of step (a) is differently modified by substitutions of 1-8 amino acids (e.g. 1, 2, 3, 4, 5, 6, 7 or 8 amino acids) in regions 2-8 and/or 62-68, preferably selected from positions 2, 4, 6, 8, 62, 63, 64, 65, 66 and 68, of SEQ ID NO: 1 or SEQ ID NO: 10.

In preferred embodiments of the eleventh aspect, the target molecule is a tumor antigen. In particularly preferred embodiments, the target molecule is the extradomain B (ED-B) of fibronectin.

In one embodiment of the eleventh aspect, the fusion protein produced in step (j) is in the form of inclusion bodies. In a further preferred embodiment of the eleventh aspect, the method further comprises the steps: isolating the inclusion bodies; solubilizing said inclusion bodies, thereby obtaining soluble fusion proteins; and further purifying the soluble fusion proteins obtained in the preceding step by at least two chromatographic steps. Suitable chromatographic steps include without limitation size-exclusion chromatography, anion exchange chromatography and cation exchange chromatography.

Optionally, the modification may be performed by genetic engineering on the DNA level and expression of the modified protein in prokaryotic or eukaryotic organisms or in vitro. In a further embodiment, the modification includes a chemical synthesis step.

In one embodiment, said population of differently modified proteins is obtained by genetically fusing two DNA libraries encoding each for differently modified monomeric ubiquitin proteins.

In another embodiment, the fusion protein of the invention can further be prepared by chemical synthesis.

### Methods of Mutagenesis of Ubiquitin

By way of example, the cDNA of ubiquitin, which can be prepared, altered, and amplified by methods known to those skilled in the art, can be used as a starting point for the mutagenesis of the respective sequence segments. For site-specific alteration of ubiquitin in relatively small regions of the primary sequence (about 1-3 amino acids) commercially available reagents and methods are on hand (“Quick Change”, Stratagene; “Mutagene Phagemid in vitro Mutagenesis Kit”, Bio-Rad). For the site-directed mutagenesis of larger regions specific embodiments of e.g. the polymerase chain reaction (PCR) are available to those skilled in the art. For this purpose a mixture of synthetic oligodeoxynucleotides having degenerated base pair compositions at the desired positions can be used for example for the introduction of the mutation. This can also be achieved by using base pair analogs which do not naturally occur in genomic DNA, such as e.g. inosine.

Starting point for the mutagenesis can be for example the cDNA of ubiquitin or also the genomic DNA. Furthermore, the gene coding for the ubiquitin protein can also be prepared synthetically.

Different procedures known per se are available for mutagenesis, such as methods for site-specific mutagenesis, methods for random mutagenesis, mutagenesis using PCR or similar methods.

In a preferred embodiment of the invention the amino acid positions to be mutagenized are predetermined. The selection of amino acids to be modified is carried out to meet the predetermined limitations with respect to those amino acids which have to be modified. In each case, a library of different mutants is generally established which is screened using methods known per se. Generally, a pre-selection of the amino acids to be modified can be particularly easily performed as sufficient structural information is available for the ubiquitin protein to be modified.

Methods for targeted mutagenesis as well as mutagenesis of longer sequence segments, for example by means of PCR, by chemical mutagenesis or using bacterial mutator strains also belong to the prior art and can be used according to the invention.

In one embodiment of the invention the mutagenesis is carried out by assembly of DNA oligonucleotides carrying the amino acid codon NNK. It should be understood, however, that also other codons (triplets) can be used. It comprises both site-specific and random mutagenesis. Site-specific mutagenesis comprising a relatively small region in the primary structure (about 3-5 amino acids) can be generated with the commercially available kits of Stratagene® (QuickChange®) or Bio-Rad® (Mutagene® phagemid in vitro mutagenesis kit) (cf. U.S. Pat. No. 5,789,166; U.S. Pat. No. 4,873,192).

If more extended regions are subjected to site-specific mutagenesis a DNA cassette must be prepared wherein the region to be mutagenized is obtained by the assembly of oligonucleotides containing the mutated and the unchanged positions (Nord et al., 1997 Nat. Biotechnol. 8, 772-777; McConell and Hoess, 1995 J. Mol. Biol. 250, 460-470.). Random mutagenesis can be introduced by propagation of the DNA in mutator strains or by PCR amplification (error-prone PCR) (e.g. Pannekoek et al., 1993 Gene 128, 135 140). For this purpose, a polymerase with an increased error rate is used. To enhance the degree of the mutagenesis introduced or to combine different mutations, respectively, the mutations in the PCR fragments can be combined by means of DNA shuffling (Stemmer, 1994 Nature 370, 389-391). A review of these mutagenesis strategies with respect to enzymes is provided in the review of Kuchner and Arnold (1997) TIBTECH 15, 523-530. To carry out this random mutagenesis in a selected DNA region also a DNA cassette must be constructed which is used for mutagenesis.

Random modification is performed by methods well-established and well-known in the art. In order to introduce the randomized fragments properly into the vectors, it is according to the invention preferred that the random nucleotides are introduced into the expression vector by the principle of site-directed PCR-mediated mutagenesis. However, other options are known to the skilled person, and it is e. g. possible to insert synthetic random sequence libraries into the vectors as well.

To generate mutants or libraries by fusion PCR, for example three PCR reactions may be carried out. Two PCR reactions are performed to generate partially overlapping intermediate fragments. A third PCR reaction is carried out to fuse the intermediate fragments.

The method for construction the library or mutant variants may include constructing a first set of primers around a desired restriction site (restriction site primer), a forward and reverse restriction primer and a second set of primers around, e.g., upstream and downstream of the codon of interest (the mutagenic primers), a forward and reverse mutagenic primer. In one embodiment, the primers are constructed immediately upstream and downstream respectively of the codon of interest. The restriction and mutagenic primers are used to construct the first intermediate and second intermediate fragments. Two PCR reactions produce these linear intermediate fragments. Each of these linear intermediate fragments comprises at least one mutated codon of interest, a flanking nucleotide sequence and a digestion site. The third PCR reaction uses the two intermediate fragments and the forward and reverse restriction primers to produce a fused linear product. The opposite, heretofore unattached ends of the linear product are digested with a restriction enzyme to create cohesive ends on the linear product. The cohesive ends of the linear product are fused by use of a DNA ligase to produce a circular product, e. g. a circular polynucleotide sequence.

To construct the intermediate fragments, the design and synthesis of two sets of forward and reverse primers are performed, a first set containing a restriction enzymes digestion site together with its flanking nucleotide sequence, and the second set contains at least one variant codon of interest (mutagenic primers). Those skilled in the art will recognize that the number of variants will depend upon the number of variant amino acid modifications desired. It is contemplated by the inventor that if other restriction enzymes are used in the process, the exact location of this digestion site and the corresponding sequence of the forward and reverse primers may be altered accordingly. Other methods are available in the art and may be used instead.

It is often necessary to couple the random sequence to a fusion partner, for example TNFalpha monomers, by having the randomized nucleotide sequence fused to a nucleotide sequence encoding at least one fusion partner. Such a fusion partner can e. g. facilitate expression and/or purification/isolation and/or further stabilization of the expression product or may involve other favorable effects.

Selection of the Modified Ubiquitin Proteins with Binding Affinity with Respect to the Cancer Target Molecule (e.g. ED-B) and Determination of the Modified Amino Acids Responsible for the Binding Affinity

After e.g. at least two different DNA libraries encoding for hetero-dimeric modified ubiquitin proteins have been established by differently modifying selected amino acids in each of the monomeric ubiquitin units, these libraries are genetically fused by e. g. linker technology to obtain DNA molecules encoding for hetero-dimeric modified ubiquitin proteins. The DNA of these libraries is expressed into proteins and the modified dimeric proteins obtained thereby are contacted according to the invention with the tumor target molecule (e.g. a tumor antigen such as ED-B) to optionally enable binding of the partners to each other if a binding affinity does exist. The contacting and screening process is performed already with respect to the hetero-dimeric ubiquitin protein. This process enables screening on those ubiquitin proteins which provide a binding activity to its target molecule. See, for example, WO 2011/073214, WO 2011/073208, and WO 2011/073209 for more details of the selection method. The contents of WO 2011/073214, WO 2011/073208, and WO 2011/073209 are herewith incorporated by reference.

Contacting according to the invention is preferably performed by means of a suitable presentation and selection method such as the phage display, ribosomal display, mRNA display or cell surface display, yeast surface display or bacterial surface display methods, preferably by means of the phage display method. For complete disclosure, reference is made also to the following references: Hoess, Curr. Opin. Struct. Biol. 3 (1993), 572-579; Wells and Lowmann, Curr. Opin. Struct. Biol. 2 (1992), 597-604; Kay et al., Phage Display of Peptides and Proteins—A Laboratory Manual (1996), Academic Press. The methods mentioned above are known to those skilled in the art and can be used according to the invention including modifications thereof.

The determination whether the modified protein has a quantifiable binding affinity with respect to a predetermined binding partner can be performed according to the invention preferably by one or more of the following methods: ELISA, plasmon surface resonance spectroscopy, fluorescence spectroscopy, FACS, isothermal titration calorimetry and analytical ultracentrifugation.

### Uses of Preferred Fusion Proteins of the Invention

The fusion proteins of the invention, which preferably comprise a modified ubiquitin heterodimer specific for ED-B and a scTNFalpha molecule, are to be used for instance for preparing therapeutic means. The fusion proteins according to the invention can be used e.g. as direct effector molecules. Examples of tumors with abundant appearance of ED-B antigen are shown in the Table 2.

TABLE 2
Occurrence of ED-B in Tumors

| Cancer | References (examples) |
|---|---|
| Renal cell | Johannsen et al. 2010, Eur J Cancer 46(16): 2926-2935 |
| Melanoma | Frey et al. 2011 Exp Dermatol 20(8): 685-8. |
| Lymphoma | Schliemann et al. 2009, Leuk Res 33(12): 1718-1722 |
| Breast | Midulla et al. 2000 Cancer Res 60(1): 164-169 |
| Colorectal | Midulla et al. 2000 Cancer Res 60(1): 164-169 |
| Head and Neck | Birchler et al. 2003 Laryngoscope 113(7): 1231-1237 |
| Hepatocellular | Menrad and Menssen 2005 Expert Opin Ther Targets |
| | 9(3): 491-500 |
| Lung | Pedretti 2009 Lung Cancer. 64(1): 28-33 |
| Osteosarcoma | Kilian et al. 2004 Bone 35(6): 1334-1345. |
| Pancreas | Wagner et al. 2008 Clin Cancer Res 14(15): 4951-4960 |
| Prostate | Berndt et al. 2010 Histochem Cell Biol 133(4): 467-75 |

Depending on the selected fusion partner the pharmaceutical composition of the invention is adapted to be directed to the treatment of cancer or any other tumor diseases, for example, in which ED-B is abundant, such as the tumors listed in Table 2.

The compositions are adapted to contain a therapeutically effective dose. The quantity of the dose to be administered depends on the organism to be treated, the type of disease, the age and weight of the patient and further factors known per se.

The compositions contain a pharmaceutically acceptable carrier and optionally can contain further auxiliary agents and excipients known per se. These include for example but are not limited to stabilizing agents, surface-active agents, salts, buffers, coloring agents etc.

The pharmaceutical composition can be in the form of a liquid preparation, a cream, a lotion for topical application, an aerosol, in the form of powders, granules, tablets, suppositories, or capsules, in the form of an emulsion or a liposomal preparation. In particular, a combination of different compositions can be used, for example applying the fusion protein of the invention in the form of a liquid preparation, a cream, a lotion for topical application, an aerosol, in the form of powders, granules, tablets, suppositories, or capsules, in the form of an emulsion or a liposomal preparation and the chemotherapeutics as liposomal preparation. The compositions are preferably sterile, non-pyrogenic and isotonic and contain the pharmaceutically conventional and acceptable additives known per se. Additionally, reference is made to the regulations of the U.S. Pharmacopoeia or Remington's Pharmaceutical Sciences, Mac Publishing Company (1990).

In the field of human and veterinary medical therapy and prophylaxis pharmaceutically effective medicaments containing at least a fusion protein in accordance with the invention can be prepared by methods known per se. Depending on the galenic preparation these compositions can be administered parenterally by injection or infusion, systemically, rectally, intraperitoneally, intramuscularly, subcutaneously, transdermally or by other conventionally employed methods of application. The type of pharmaceutical preparation depends on the type of disease to be treated, the severity of the disease, the patient to be treated and other factors known to those skilled in the art of medicine.

It surprisingly turned out that a fusion protein of a targeting moiety, e.g. a modified ubiquitin hetero-dimer with binding affinity to a cancer target (ED-B) fused to scTNFalpha, wherein the fusion protein preferably has a sequence selected from the group consisting of SEQ ID NOs: 17 to 22, can be advantageously applied in therapy. This approach provides a less toxic, but still therapeutically effective concentration. Since scTNFalpha is coupled to a highly specific targeting moiety, it can be directly active at the tumor.

Thus, systemic side effects of TNFalpha can be remarkably reduced by administering a fusion protein according to the present invention. By using a fusion protein of the invention, the overall dosage of TNFalpha to reach a therapeutic effect can thus be reduced to a large extent and can be advantageously used for systemic tumor treatment in particular in combination with chemotherapeutic agents. The fusion protein of the invention can be used as tumor targeted effective therapeutic drug in combination with a cytotoxic drug. In an embodiment, the pharmaceutical composition contains a fusion protein of the invention or a combination thereof and further comprises one or more chemotherapeutic agents, preferably selected from the following table:

TABLE 3
Chemotherapeutic agents for pharmaceutical
compositions with fusion protein of the invention

| Substance Class | Examples |
|---|---|
| Alkylating agents (ATC | melphalan, cyclophosphamide (e.g. Endoxan) |
| L01A) | |
| Intercalating agents | doxorubicin (Myocet), pegylated liposomal |
| (ATC L01DB) | doxorubicin (Lipodox, Caelyx), Idarubicin, |
| | Daunorubicin |
| Antimetabolites (ATC | 5-fluorouracil, gemcitabine, cytarabin |
| L01B) | |
| Taxanes (ATC L01CD) | Paclitaxel, Docetaxel |
| Cytotoxic antibiotics | Dactinomycin |
| (ATC L01D) | |
| Platinum analoga | Cisplatin |
| (ATC L01XA) | |
| Mitosis inhibitors | Vincristin, vinblastin |
| (ATC L01CA02) | |
| Topoisomerase | Irinotecan |
| Inhibitor (ATC L01XX) | |

In a preferred embodiment, the chemotherapeutic agent (cytostatics) is selected from alkylating agents, platinum analogs, antibiotics, taxanes, intercalating agents (e.g. anthracyclines), anti-metabolites, mitosis inhibitors and topoisomerase inhibitors, consisting of, for example, Melphalan, Lipodox, doxorubicin, cyclophosphamide, dactinomycin, fluorodesoxyuracil, 5-fluorouracil, cisplatin, paclitaxel, gemcitabine, Docetaxel, Folfox/Folfiri, Carboplatinum, Pemetrexed, Irinotecan, Capecitabin, Vinorelbin, Epirubicin, Mitoxantron or radiopharmaceuticals or nanoparticular formulations of cytostatics (e.g. Doxil and Abraxane) and others and adjuvants. A particularly preferred combination is a fusion protein according to the invention and melphalan, and/or (liposomal) doxorubicin (for example, Lipodox). Apart from antineoplastic agents from the ATC class L01, the TNF-fusion protein of the invention can be combined with other antineoplastic substances including cytokines and derivatives thereof, radiopharmaceuticals, cell based therapeutics and nanoparticles. Due to its tumor permeabilisation activity, the TNF-fusion protein of the invention (but also the other recombinant proteins/fusion proteins of the present invention) can be combined with all antineoplastic agents as listed under L01 in the Anatomical Therapeutic Chemical Classification System (ATC) provided by the World Health Organisation.

In a further embodiment, the pharmaceutical composition is in the form of a kit of parts, providing separated entities for a fusion protein of the invention and for the one or more chemotherapeutic agents. It surprisingly turned out that a fusion protein of a tumor targeting moiety, preferably a modified ubiquitin hetero-dimer, fused to scTNFalpha can be advantageously applied in therapy (see Examples 6 and 7). Native TNFalpha is highly toxic and, thus, may only be administered in low dosages, which usually lie below the minimum therapeutic threshold (and thus are therapeutically inactive). By applying the targeted scTNFalpha fusion proteins of the present invention, it is possible to administer TNFalpha in a non-toxic, but still therapeutically effective concentration. Since a single chain TNFalpha molecule is coupled to a highly specific targeting domain, it can be directly active at the disease site (for example, tumor site) and, thus, the amount of “free” TNFalpha can be drastically reduced which reduces severe side effects in patients. Thus, it is expected that systemic side effects of TNFalpha can be remarkably reduced by administering a fusion protein according to the present invention consisting of a single chain TNFalpha and a targeting domain. By using a single chain TNFalpha fusion protein of the invention, the overall dosage to reach a therapeutic effect thus is expected to be reduced and can be advantageously used for systemic tumor treatment (without the necessity and restrictions of limb perfusion), in particular if used in combination with chemotherapeutic agents (see above). Administration of a fusion protein consisting of anti-EDB Affilin and scTNFalpha results in tumor accumulation of the cytokine. This is achieved by enhancing vessel permeabilization and thereby improving cytostatic accumulation in the tumor.

The fusion protein is targeted by the targeting moiety specifically and with high affinity to the tumor tissue. At the tumor side, the biological function of TNFalpha enables improved permeability of the tumor tissue which is important for the action of the chemotherapeutic agent directly at the side of the tumor.

In a further embodiment, the pharmaceutical composition is in the form of a kit of parts, providing separated entities for the fusion protein of the invention and for the one or more chemotherapeutic agents.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1

### Expression of Affilin-scTNF Fusion Proteins

The fusion proteins studied in the following examples consist of a modified hetero-dimeric ubiquitin as targeting domain and at least three C-terminally fused mammalian (mouse or human) TNFα monomers (scTNFalpha). In one embodiment, fusion proteins were produced as inclusion bodies in E. coli and purified after in vitro refolding. By way of characterization, the obtained fusion protein preparations were analyzed for purity and homogeneity. TNFα activity in cell culture, affinity for target protein ED-B, selectivity, and specific binding in cell culture were tested.

### Step 1: Production of a Vector for Cloning of Affilin-scTNF-Fusion Proteins

As vector for cloning of fusion proteins, proprietary expression vectors (Scil Proteins GmbH, pSCIL008b, see WO 05/061716) or commercially available vectors (e.g., pET20a or pET20b by Invitrogen) were modified by insertion of coding sequences for three TNFalpha monomers joined by linker peptides (e.g. (GGGGS)₃ (=SEQ ID NO: 8) or (GGGS)₃ (=SEQ ID NO: 9)), corresponding to a single-chain TNFalpha moiety. The scTNFalpha sequence was amplified via PCR. Unique restriction sites were introduced into the resulting expression plasmids in order to facilitate the insertion of modified ubiquitin sequences.

### Step 2: Cloning of Affilin-scTNF-Fusion Proteins

For the production of the fusion proteins of ED-B-binding modified ubiquitin-based variants and scTNFalpha, the sequence of interest encoding for ED-B-binding modified ubiquitin was amplified from a plasmid template by PCR according to standard procedures, and inserted into the expression plasmids described in step 1. DNA sequence analyses confirmed the correct sequences of the expression vectors encoding the fusion proteins.

### Step 3. Expression of Affilin-scTNFalpha-Fusion Proteins

64177 (murine scTNFalpha fusion with heterodimeric ubiquitin) or 75808 (human scTNFalpha fusion with heterodimeric ubiquitin) or other fusion proteins were produced in E. coli and isolated in the form of inclusion bodies. For expression of the fusion proteins, the clones were cultivated and grown by fed-batch fermentation in complex media containing the appropriate antibiotics corresponding to the respective expression vectors. Expression was induced by adding IPTG. After 2-4 h of induction, microbial cells were harvested, suspended, and disrupted by high pressure dispersion in a French press. Although both heterodimeric ubiquitin molecules and scTNFalpha are soluble proteins if not expressed as fusion proteins, the fusion proteins comprising the Affilin moiety and the scTNF moiety, for example 64177 and 75808, are surprisingly expressed in insoluble form. Both fusion proteins 64177 and 75808 are almost exclusively found in the insoluble fractions (see lane 4 in FIG. 3A and lane 4 in FIG. 3B) and not in the soluble fractions (see lane 5 of FIG. 3A and lane 5 of FIG. 3B). The insoluble fraction was collected and inclusion bodies containing the expressed proteins were isolated by standard washing protocols.

### Example 2

### In Vitro Refolding and Purification of Affilin-scTNFalpha Fusion Proteins

Active fusion proteins were prepared by in vitro refolding at a temperature of 4 degrees centigrade after rapid dilution of inclusion body material solubilized in 6 M guanidinium chloride into phosphate-buffered saline (or other appropriate buffer system) and purified by a series of chromatographic steps. For fusion proteins containing scTNFalpha derived from murine TNFalpha (64177), these chromatographic steps included anion exchange chromatography on, e.g. Q Sepharose HP, followed by size exclusion chromatography on a Superdex™ 200 pg column. For fusion proteins containing scTNFalpha derived from human TNFalpha (75808), anion exchange chromatography was replaced by a cation exchange step on, e.g, SP Sepharose HP. In all cases, fractions were analyzed by SDS-PAGE and analytical HPLC with respect to their purity. Suitable fractions were pooled and analyzed for homogeneity and activity by a series of methods including, e.g., rpHPLC, SE-HPLC, analytical affinity interaction chromatography, and surface plasmon resonance-based interaction analysis (see FIGS. 3 C to 3 F). For fusion protein 64177, yields of up to 34 mg active fusion protein per liter expression culture from fed-batch fermentation were obtained. Yields obtained for other fusion proteins of EDB-binding modified ubiquitin-based variants (Affilin) and mouse scTNFalpha were comparable. For example, after batch cultivations in shaker flasks, 22 mg and 12.2 mg of purified and active fusion proteins 83563 and 83564, respectively, were obtained per liter expression culture. For 75808, 7.9 mg purified active fusion protein were obtained per liter expression culture after batch cultivation in shaker flasks.

### Example 3

### Analysis of TNFalpha Activity of Fusion Proteins

### Example 3A

### L929 Apoptosis Assay

The physiological TNFalpha-activity of an Affilin-scTNF-fusion protein has been determined using the L929 apoptosis assay (Flick et al., 1984 J. Immunol. Methods. 68:167-175). In this assay, the effector part of the fusion protein (scTNFalpha, three soluble TNFalpha monomers connected via peptide linkers) efficiently stimulates cell death in actinomycin D sensitized cells at EC₅₀ values in the picomolar range.

Cells were resuspended in suitable medium containing FBS and antibiotics. A cell suspension of a density of 3.5×10⁵ cells/ml in medium containing 2% FCS was seeded into the wells of a 96 well standard cell culture plate. After incubation, the culture medium was removed and medium containing 4% FBS, Actinomycin D and antibiotics was added to each well. After incubation, a fusion protein of the invention or the TNFalpha control were added at appropriate concentration ranges (e.g. 5×10⁻¹¹ and 10⁻¹⁵ M). After a further incubation of 24 h, the metabolic activity as a measure of cell survival was determined using WST-1 reagent (Roche). Per test item at least two independent experiments were conducted, each of them in duplicates. Each testing of fusion proteins of the invention was paralleled by testing a dose range of murine recombinant TNFalpha to validate the assay. The quantitative evaluation is based on the relative potency against a mTNFalpha-standard.

FIG. 4A shows the activity of the scTNFalpha part of fusion proteins 64179, 64177, 83563 (SEQ ID NO: 19; identical to 85653) and 83564 (SEQ ID NO: 21; identical to 83463 and to 85654) by L929 cytotoxicity assay. In this embodiment, an L929 assay was used as activity assay versus mouse TNFalpha as internal standard (mTNFalpha in the figure). The figure shows that purified fusion proteins have at least the same activity compared to the mouse TNFalpha. Thereby, the scTNFalpha moiety within the fusion proteins shows nearly the same activity as the free TNFalpha. This result is particularly surprising, when considering the fact that the fusion proteins were refolded from inclusion bodies. Recombinant scTNFalpha described in the prior was always produced as a soluble protein (see for example Krippner-Heidenreich et al., supra). Moreover, fusion of different hetero-dimeric ubiquitin (Affilin) variants to the N-terminus of scTNFalpha does not interfere with its in vitro activity. Fusion proteins comprising scTNFalpha and hetero-dimeric ubiquitin variants have not been described in the prior art. Prior to the experiments described herein, it could not be predicted whether the presence of the hetero-dimeric ubiquitin variants would interfere with the correct folding and trimerization of the TNFalpha monomers or not. Potency has been determined in several independent assays and is determined by parallel line method with PLA2.0 software relative to the internal control mTNFalpha. The potency of mTNFalpha should be in a range of 100%+/−20%.

### Example 3B

### NF-kappaB-RE assay

The TNFalpha activity of the fusion proteins was also detected by an NF-kappaB-response element (RE) assay. NF-kappaB (nuclear factor “kappa-light-chain-enhancer” of activated B-cells) is a heterodimeric transcription factor present in all cells and tissues. NF-kappaB-response element (RE) is a DNA-binding sequence for the NF-kappaB transcription factor that is responsible for immune response, cell growth and apoptosis. For these investigations, the pGL4.32[/uc2P/NF-kB-RE/Hygro] vector obtained from Promega was used. It contains five copies of NF-kappaB-RE that drives the transcription of the luciferase reporter gene luc2P (Photinus pyralis).

The pGL4.32[luc2P/NF-kB-RE/Hygro] vector was stably transfected in HeLa-cells, a human immortal cervical cancer cell line. The stable cell pool HeLa/pGL4.32[luc2P/NF-kB-RE/Hygro] was selected by 400 μg/ml Hygromycin.

For measurement of TNFalpha-activity, 3×10⁴ cells/well (corresponding to 3×10⁵ cells/ml) were seeded into wells of a 96-well-plate in medium containing 5% FCS (fetal calf serum) and gentamicin. Cells were incubated at 37° C., 5% CO₂ and 95% humidity for 24 hours. Thereafter, cells were treated with fusion proteins or TNFalpha [1e⁻⁹ to 1.69e⁻¹⁴ M] in triplicates for 5 h at 37° C. In order to determine the TNFalpha activity, cells were incubated with ONE-Glo™ Luciferase substrate from Promega for 10 min at room temperature. The luminescence signal was read out by plate reader.

FIG. 4B shows the activity of the scTNFalpha part of fusion proteins 83564 and 64179 by the NFkappaB-RE-assay with mouse TNFalpha as internal standard. The figure shows that purified fusion protein 83564 has even higher activity compared to the mouse TNFalpha. No loss of TNF-alpha activity was observed in different activity assays. Thereby, the scTNFalpha moiety within the fusion protein 83564 shows better activity as the free TNFalpha. Fusion of different hetero-dimeric ubiquitin variants to the N-terminus of scTNFalpha does not interfere with its activity. As explained above in Example 3A, these results are particularly surprising, when considering the fact that the fusion proteins were refolded from inclusion bodies. In addition, it could not be predicted whether the presence of the hetero-dimeric ubiquitin variants would interfere with the correct folding and trimerization of the TNFalpha monomers or not.

### Example 4

### Activity of the Hetero-Dimeric Ubiquitin-Based Binding Domain in the Fusion Proteins

### Example 4 A

### Binding Analysis of Fusion Proteins 64177, 83563 and 83564 to Human ED-B by ELISA

In order to analyze the target binding of the fusion proteins, a human ED-B construct was coated to Nunc microwell plates in a concentration of 5 μg/ml. Unspecific binding sites were blocked with BSA blocking solution in PBS. After washing the wells with PBST buffer, fusion protein was applied in a concentration series of 0-100 nM. The wells were again washed with PBST. For detection of immobilized fusion proteins containing scTNF, a commercially available construct comprising the soluble TNF-binding domain of TNF receptor I and the Fc portion of human IgG (R&D systems) was employed. After incubation and an additional washing step, a POD-conjugate of Fc-specific anti human IgG was applied in order to label the bound TNF receptor chimera. Specific binding of the receptor chimera to the immobilized fusion proteins was monitored by a POD-catalyzed colorimetric reaction using the substrate 3,3′,5,5′ tetramethylbenzidin (KEM-EN-Tec) according to the manufacturer's instructions. Reactions were stopped by adding 0.2 M H₂SO₄. The ELISA plates were read out using the TECAN Sunrise ELISA-Reader. The photometric absorbance measurements were done at 450 nm using 620 nm as a reference wavelength.

For all fusion proteins containing scTNF derived from mammalian TNFalpha that were tested, strong specific target binding was observed (K_{D} 0.3-0.4 nM). FIGS. 5 A-C shows the binding of fusion proteins 64177, 83563 and 83564 to immobilized human ED-B, analyzed by ELISA. The K_{D} determined by least-square fit of the date was 0.33 nM for 64177, 0.42 nM for 84563 and 0.39 nM for 84564.

### Example 4 B

### Binding Analysis of Fusion Proteins 64177 and 75808 to Human ED-B by Biacore Assays

Concentration series (e.g., 0-200 nM) of purified fusion proteins were analyzed for binding to human and mouse ED-B by surface plasmon resonance experiments using a Biacore™ instrument. ED-B constructs were immobilized on SA sensor chips via N-terminally introduced biotin using methods known to those skilled in the art.

For binding, e.g., of the fusion proteins to human and murine ED-B, equilibrium dissociation constants (K_{D})<10⁻⁹M were determined for all purified preparations.

FIGS. 5 D and 5 E show a surface plasmon resonance (Biacore™) analysis of the human ED-B binding activity of the modified ubiquitin moiety within fusion proteins 64177 and 75808. The equilibrium dissociation constant K_{D} determined from a global least-square fit of the data was 0.22 nM for 64177, while the determined rate constants were 6.12*10⁵ M⁻¹s⁻¹ for association and 1.36*10⁴s⁻¹ for dissociation, respectively. The equilibrium dissociation constant K_{D} determined from a global least-square fit of the data (dashed line) was 0.12 nM for 75808, while the determined rate constants were 1.33*10⁶ M⁻¹s⁻¹ for association and 1.62*10⁻⁴s⁻¹ for dissociation, respectively.

### Example 5

### Binding Analysis of the Affilin-scTNF-Fusion Protein on Cells

### Example 5A

### Binding Analysis of Fusion Proteins 64177, 83563, and 83564 on Vital Fibroblast Cells

ED-B is expressed in tumors and the matrix on embryonic cells. The binding of the cancer target-binding protein fused to scTNFalpha to the matrix of cell culture cells was compared to the binding of the control and non-targeting domain protein fused to scTNFalpha (64179). Different cell culture cells were analyzed, including normal human fetal lung fibroblast cells having high expression levels of ED-B (Wi38 cells), and normal human dermal fibroblasts having low level of ED-B (NHDF). To analyze the in vitro binding, 64177, 83563, 83564 and 64179 were investigated on vital Wi38 cells.

Fusion proteins (single concentration of 1, 5 and 10 nM) or control protein 64179 (1, 5 and 10 nM) were incubated (1 h, 37° C.) with Wi38 and NHDF cells (60,000 cells/ml; from ATCC), followed by fixation with methanol, blocking (5% Horse serum/PBS); incubation with an anti-TNFalpha-antibody (Peprotech 500-P64, 1:500) and additionally with a secondary Alexa488 conjugated antibody (obtained from Invitrogen A11008, 1:1000). The nuclei were stained with DAPI. The photometric documentation was generated by merging of a green fluorescent (480 nm) image and an image, which shows only the nuclei staining (350 nm).

The analysis clearly shows that the fusion proteins binds to vital Wi38 cells with high specificity to ED-B containing extracellular matrix (see FIG. 6 A). Lane 1 shows a strong EDB-staining of 83563, 83564 and 64177 on Wi38 cells. No unspecific staining was observed with the non-binding protein 64179 or the PBS control (row 4, 5). The second lane shows the analyses of scTNFalpha-fusion proteins on human normal dermal fibroblast cells. On these low level EDB-expressing cells only weak binding has been observed for the scTNFalpha fusion proteins.

The negative control cell type NHDF are primary normal fibroblast cells, which express low levels of EDB-fibronectin, corresponding to a reduced staining pattern by the fusion proteins of the invention.

### Example 5B

### Binding Analysis of Fusion Proteins 64177, 83563, and 83564 on F9 Tumor Cells

ED-B accumulates around neovascular structures, like tumor blood vessels (Tarli et al., 1999, Blood 94: 192-198). Different concentrations of fusion proteins were compared with respect to ED-B target binding on F9 teratocarcinoma slides. Slices of a thickness of 6 μm were fixed with ice cold absolute acetone. After blocking with 5% horse serum, slices were incubated with 1, 5 and 10 nM of fusion proteins 64177, 83563, 83564 and the non-ED-B targeted protein 64179, respectively. Fusion proteins were probed by anti-TNFalpha-antibody (Peprotech 500-P64, 1:500) and additional with a secondary Alexa488 conjugated antibody (Invitrogen, A11008, 1:1000). CD31 (PECAM-1) is a widely used endothelial cell marker. Therefore, vessels were stained by use of an anti-mouse CD31-antibody (10 μg/ml; Abcam, ab56299) and an Alexa594-conjugated secondary antibody.

FIG. 6 B shows a specific binding of fusion proteins 64177, 83563 and 83564 at 1 nM, 5 nM and 10 nM concentrations on F9-tumor tissue. Staining of F9 slices by the non-binding protein 64179 was not detected.

FIG. 6 C shows the accumulation of the fusion proteins close to F9-tumor vessels. In particular, the figure shows the individual staining as well as co-staining of the EDB-target and tumor vessels as determined by use of 1 nM fusion protein (64177, 83563, 83564, and 64179, respectively). Tumor vessels were represented by anti-CD31-staining. The merged pictures showed the association of EDB-binding proteins with the vessels of the F9-tumor. It is demonstrated that all fusion proteins provide a predominant vessel association whereas the non-targeting fusion protein does not show any staining on the slices.

### Example 6

### In Vivo Efficacy Study in Tumor Bearing Mice Using Fusion Protein 64177

To establish the therapeutic efficacy of a scTNFalpha-EDB-binding fusion protein, tumor-bearing mice were treated with a combination of the control fusion protein or an EDB-binding fusion protein each with a chemotherapeutic drug selected from the substance class ATC L01 (alkylating agents). For this study, Melphalan was used as exemplary cytotoxic drug; any other cytotoxic agents could be used instead. As negative control, only a buffer was injected into tumor-bearing mice, with and without the addition of Melphalan.

The fusion protein was tested in a mouse model of F9 teratoma (see Borsi et al., 2003 Blood 102, 4384-4392). F9 mouse embryonal teratocarcinoma cells were provided by Cell Line Services (cat. No. 400174). The ED-B expression of the F9 tumors is strongly upregulated as shown in the human in vivo situation of numerous cancer entities. The model is therefore suitable for an evaluation of the therapeutic impact of the fusion protein of the invention on cancer, e.g. in combination with a cytotoxic compound such as Melphalan. F9 teratoma is an aggressive tumor with high vascular density. Borsi et al. described that targeting of mouse TNFalpha via EDB-antibodies improves the efficacy of Melphalan which is demonstrated by retardation of tumor growth. The experimental schedule for the efficacy study was adapted from Borsi, 2003. The cells were grown until reaching 50-70% confluence. 1×10⁶ viable cells were subcutaneously implanted into female 8 week old immunocompetent syngeneic 129S mice (The Jackson Laboratories). For treatments, groups of n=8 tumor-bearing mice with a tumor size between 60 and 150 mm³ were intravenously injected into the tail vein either with 64177, the negative control protein 64179 or PBS in a total applied volume of 10 ml/kg. Doses of 2.1 pmol/g, 0.7 pmol/g and 0.23 pmol/g were used for the EDB-binding fusion protein and the control fusion protein without tumor targeting domain. 24 h later Melphalan (Alkeran® 50 i.v.) were intraperitoneally injected at a dose of 4.5 mg/kg. Animal body weight and tumor volume was determined in 24 h intervals following the first treatment. Daily observations of the animals regarding potential clinical signs were performed. Animals with tumor ulceration, body weight loss of >20% and tumor weight>10% of body weight were excluded from the study.

FIG. 7 shows the relative tumor growth during the time of observation (10 days). The figure clearly shows that the EDB-binding fusion protein with effector and targeting domain in combination with Melphalan reduces the relative tumor growth more efficiently than the control fusion protein in combination with Melphalan or than Melphalan alone. The tumor growth kinetics 10 days after treatment shows the reduction of tumor volume by the fusion protein comprising a scTNFalpha molecule and a tumor targeting domain. FIG. 6 shows tumor growth curves of animals treated with PBS (closed circles, black), PBS plus Melphalan (open squares, black), 64177 plus Melphalan (open circles, black) and 64179 plus Melphalan (open triangles, black) plus Melphalan. FIG. 7 shows that the scTNFalpha-EDB-binding fusion protein of the invention (64177) delayed the tumor growth compared to the control protein without scTNFalpha (effector domain) as well as vehicle (PBS) and Melphalan control groups by 2 and 4 days, respectively. This is a clear evidence for efficacy in combination with Melphalan.

### Example 7

### In Vivo Toxicity Study Using Affilin-scTNF-Fusion Protein 64177

To establish the toxic effect of a scTNFalpha-EDB-binding fusion protein, naïve CD1 mice were intravenously treated with 64177 at doses of 650 μg/kg, 180 μg/kg or 50 μg/kg, respectively. As reference item mouse TNFalpha (mTNFalpha) at a dose of 470 μg/kg (=equimolar to the highest 64177 dose of 650 μg/kg) was injected intravenously into separate CD1 mice as well. Two groups of 3 male and 3 female mice for each treatment dose were recruited. One group of 3 male and 3 female mice for each treatment dose was sacrificed 24 h after the intravenous administration to obtain information on acute TNFalpha mediated effects. The second group of each treatment dose was sacrificed 14 days after intravenous administration to provide information on the recovery of the obtained acute effects. Parameters that were analyzed are: clinical signs (local tolerance, systemic tolerance, mortality, behavior, external appearance, faeces), body weight, body temperature, food consumption, hematology, clinical chemistry, macroscopic post mortem findings, organ weights and histopathology of dedicated organs.

The overall analysis of all data shows that the findings produced by 64177 or mTNFalpha were comparable but the incidence and/or severity of effects were generally more pronounced in the mTNFalpha-treated animals compared to the 64177-treated animals. It appears that recovery from toxic effects after the single high dose of 64177 compared to the equimolar dose of mTNFalpha occurred earlier in surviving animals. Blood chemistry data indicate reversible impairment of liver cell integrity and possible effects on other organs at dose levels that were associated with mortality. The effects on laboratory diagnostic parameters were less pronounced after administration of 650 μg/kg 64177 than after administration of the equimolar dose of mTNFalpha.

The single intravenous dose of 50 μg/kg 64177 can be considered to be the no-observed-adverse-effect level (NOAEL). At this dose, there were no signs of toxicity, lab diagnostic changes or relevant pathological findings.

The single intravenous dose of 180 μg/kg 64177 produced transient clinical signs and slight, reversible lab diagnostic changes. There were no histopathological findings at this intermediate dose level indicating a toxic effect of 64177.

### Example 8

### In Vivo Efficacy Study Using 83564 Fusion Protein in a Breast Cancer Model

The efficacy study is intended to analyze the effect of targeted fusion protein 83564 combined with a chemotherapeutic on the tumor size in a breast cancer model. As model system for the in vivo efficacy study, the breast cancer cell line MDA-MB 231 (ATCC® HTB-26™; patient derived model, obtained from ATCC) was used. Cells were implanted subcutaneously in the left flank of female immunodeficient NMRI nu/nu mice from Harlan. For this study, Lipodox was used as exemplary cytotoxic drug; any other cytotoxic agents could be used instead. The chemotherapeutic Lipodox from Sun Pharma Global FZE is composed of doxorubicin hydrochloride encapsulated in long circulating pegylated liposomes. It is indicated as monotherapy for the treatment of metastatic breast cancer with increased cardiac risk.

In order to show the efficacy of Affilin-scTNFalpha-fusion protein 83564, mice carrying an MDA-MB 231 tumor with an initial size between 50 mm³ and 200 mm³ were intravenously treated with 75 μg/kg/day 83564 combined with Lipodox at a dose of 1.5 mg/kg/day (closed squares in FIG. 8). As control substance, 75 μg/kg/day 64179 (Affilin-scTNFalpha-fusion protein without targeting) combined with Lipodox at a dose of 1.5 mg/kg/day was injected intravenously into separate mice (closed circles in FIG. 8). Furthermore, three control groups treated with vehicles and/or Lipodox alone were included into the study: (i) a vehicle control (control vehicle 1: control for the fusion proteins, consisting of 1×PBS, pH 7.3 with 0.000075% Tween®20 and 0.148% glycerin; control vehicle 2: control for Lipodox, consisting of 5% dextrose; open diamonds in FIG. 8), (ii) a vehicle control (vehicle 1) combined with Lipodox at a dose of 1.5 mg/kg/day (open circles in FIG. 8), (iii) a vehicle control (vehicle 1) combined with Lipodox at a dose of 3 mg/kg/day (open triangle in FIG. 8). Treatment with fusion proteins 83564 or 64179 was performed on days 0, 7, 14 and 21 of the study; application of Lipodox was done 24 h after the application of the fusion protein 83564 or 64179 on days 1, 8, 15 and 22. Control groups were treated in parallel with vehicle and/or Lipodox. After the four treatments, the growth of the tumors was observed for additional 7 days. During the whole efficacy study, the relative tumor volume was analyzed.

Comparing all tested groups, the growth of the tumor is reduced in the highest manner after the treatment with the targeted EDB-binding (Affilin)-scTNF-fusion protein in combination with Lipodox (see FIG. 8).

If only control vehicle was applied, the tumors showed a strong growth and a relative tumor volume of about 1333 mm³+/−116 mm³ was obtained 28 days after the start of the treatment. At the same time point, relative tumor volumes of 655 mm³+/−75 mm³ after the application of 1.5 mg/kg/day Lipodox and 571 mm³+/−32 mm³ after the application of 3 mg/kg/day Lipodox were measured. Treatment with 83564 and 1.5 mg/kg/day Lipodox led to a relative tumor volume of 510 mm³+/−53 mm³ at day 28. Thus, the combination of 83564 and 1.5 mg/kg/day Lipodox showed a higher efficacy than 1.5 mg/kg/day or 3 mg/kg/day Lipodox alone did.

After treatment with control protein 64179 in combination with 1.5 mg/kg/day Lipodox, a relative tumor volume of 651 mm³+/−61 mm³ was measured at day 28. Control protein 64179 combined with Lipodox had the effect on growth of breast cancer as Lipodox alone. However, the combination of 83564 and Lipodox showed superior effects: it reduced of tumor growth. From this study, is concluded that the tumor reducing effect is due to the targeting effect of the targeting moiety of the fusion protein.

### Sequence Listing—Free Text Information

The sequences according to SEQ ID NOs: 1, 2, and 13-15 shown in the attached sequence listing do not contain any free text information. Nevertheless, short explanations are presented below also for these sequences.

SEQ ID NO: 1: ubiquitin
SEQ ID NO: 2: extradomain B (ED-B) of fibronectin
SEQ ID NO: 3: linker sequence
SEQ ID NO: 4: linker sequence
SEQ ID NO: 5: linker sequence
SEQ ID NO: 6: linker sequence
SEQ ID NO: 7: linker sequence
SEQ ID NO: 8: linker sequence
SEQ ID NO: 9: linker sequence
SEQ ID NO: 10: ubiquitin start sequence (F45W, G75A, G76A)
SEQ ID NO: 11: Ub2; dimer of unmodified ubiquitin start sequence with GIG linker
SEQ ID NO: 12: anti-ED-B Affilin® 54646
SEQ ID NO: 13: TNFalpha, human, monomer
SEQ ID NO: 14: TNFalpha, mouse, monomer
SEQ ID NO: 15: TNFalpha, rat, monomer
SEQ ID NO: 16: 64179, fusion protein of Ub2 and scTNFalpha, murine
SEQ ID NO: 17: 64177, fusion protein of 54646 and scTNFalpha, murine
SEQ ID NO: 18: 75808, fusion protein of 54646 and scTNFalpha, human
SEQ ID NO: 19: 83563, mutein (T2V) of fusion protein 64177
SEQ ID NO: 20: h83563, mutein (T2V) of fusion protein 75808
SEQ ID NO: 21: 83564, mutein (T2R/F63P) of fusion protein 64177
SEQ ID NO: 22: h83564, mutein (T2R/F63P) of fusion protein 75808
SEQ ID NO: 23: anti-ED-B Affilin®, mutein (T2V) of 54646 (clone ID 65137)
SEQ ID NO: 24: anti-ED-B Affilin®, mutein (T2R/F63P) of 54646 (clone ID 77404)
SEQ ID NO: 25: consensus sequence of TNFalpha
SEQ ID NO: 26: linker sequence
SEQ ID NO: 27: linker sequence
SEQ ID NO: 28: linker sequence
SEQ ID NO: 29: linker sequence

1. A fusion protein comprising:
(i) a biologically active single chain TNFalpha molecule that comprises at least three TNFalpha monomers joined by linkers; and
(ii) a targeting domain that is capable of binding to a target molecule with a specific binding affinity to the target molecule of Kd≦10⁻⁷.

2. The fusion protein according to claim 1, wherein each TNFalpha monomer is a mammalian TNFalpha.

3. The fusion protein according to claim 1, wherein:
(i) the targeting domain consists of a modified dimeric ubiquitin protein; or
(ii) the modified ubiquitin protein is a hetero-dimeric ubiquitin comprising two monomeric ubiquitin units linked together in a head-to-tail arrangement, and further wherein each monomeric ubiquitin unit in said modified hetero-dimeric ubiquitin protein is modified independently from the modifications in the other monomeric ubiquitin unit; or
(iii) each modified monomeric ubiquitin unit has an amino acid sequence identity of at least 80% to the amino acid sequence defined by SEQ ID NO: 1 or to the amino acid sequence defined by SEQ ID NO: 10;
or any combination thereof.

4. The fusion protein according to claim 3, wherein each monomeric ubiquitin unit in said modified hetero-dimeric ubiquitin protein is modified independently from the modifications in the other monomeric ubiquitin unit by substitutions of 1-8 amino acids selected from positions 2, 4, 6, 8, 62, 63, 64, 65, 66, and/or 68 of SEQ ID NO: 1 or SEQ ID NO: 10.

5. The fusion protein according to claim 1, wherein the target molecule is a tumor target molecule.

6. The fusion protein according to claim 1, wherein the modified hetero-dimeric ubiquitin protein comprises an amino acid sequence that is at least 90% identical to any of SEQ ID NOs: 12, 23, and 24.

7. (canceled)

8. The fusion protein according to claim 1, wherein said biologically active single chain TNFalpha molecule comprises three TNFalpha monomers joined by two linkers in the following order:
TNFalpha monomer—linker 1—TNFalpha monomer—linker 2—TNFalpha monomer; and further wherein linker 1 and
linker 2 are both peptide linkers consisting of 10-30 amino acids each.

9. (canceled)

10. (canceled)

11. A pharmaceutical composition comprising the fusion protein of claim 1 and a pharmaceutically acceptable carrier.

12. The pharmaceutical composition according to claim 11, further comprising one or more chemotherapeutic agents.

13. The pharmaceutical composition according to claim 12, wherein the pharmaceutical composition is in the form of a combined preparation or in the form of a kit of parts.

14. A method for the preparation of a fusion protein, said method comprising:
(a) preparing a nucleic acid encoding a fusion protein comprising:
(i) a biologically active single chain TNFalpha molecule that comprises at least three TNFalpha monomers joined by linkers; and
(ii) a targeting domain that is capable of binding to a target molecule with a specific binding affinity to the target molecule of Kd≦10⁻⁷;
(b) introducing said nucleic acid into an expression vector;
(c) introducing said expression vector into a host cell; and
(d) subjecting the host cell to culturing conditions under which a fusion protein is expressed from said expression vector,
(e) optionally isolating the fusion protein produced in step (e).
whereby the fusion protein of claim 1 is prepared.

15. A method for isolating a fusion protein that binds to a target molecule, the method comprising:
(a) providing a population of differently modified dimeric ubiquitin proteins originating from monomeric ubiquitin proteins, said population comprising dimeric ubiquitin proteins comprising two modified ubiquitin monomers linked together, preferably in a head-to-tail arrangement, wherein each monomer of said dimeric protein is differently modified;
(b) providing a target molecule as a potential ligand;
(c) contacting said population of differently modified proteins with said target molecule;
(d) identifying a modified dimeric ubiquitin protein by a screening process that binds to said target molecule with a specific binding affinity of Kd≦10⁻⁷ M;
(e) isolating said modified dimeric ubiquitin protein with said binding affinity;
(f) identifying a polynucleotide sequence encoding the modified dimeric ubiquitin protein isolated in step (e);
(g) preparing a nucleic acid molecule comprising in frame:
(1) the polynucleotide sequence identified in step (f); and
(2) a polynucleotide sequence encoding a biologically active single chain TNFalpha molecule;
(h) introducing the nucleic acid molecule prepared in step (g) into an expression vector;
(i) introducing said expression vector into a host cell;
(j) culturing the host cell under conditions wherein a protein is expressed from said expression vector, thereby producing a fusion protein comprising the modified dimeric ubiquitin protein identified in step (d) and a biologically active single chain TNFalpha molecule; and
(k) isolating the fusion protein produced in step (j).

16. The method of claim 15, wherein each monomer of said dimeric protein of step (a) is differently modified by substitutions of 1-8 amino acids at amino acid positions selected from the group consisting of positions 2, 4, 6, 8, 62, 63, 64, 65, 66 and 68 of either SEQ ID NO: 1 or SEQ ID NO: 10.

17. The method of claim 15, wherein the target molecule is an extradomain B (ED-B) of fibronectin.

18. The fusion protein according to claim 1, further comprising a linker linking the biologically active single chain TNFalpha molecule and the targeting domain.

19. The fusion protein according to claim 18, wherein the linker linking the biologically active single chain TNFalpha molecule and the targeting domain is a peptide linker of 10-30 amino acids.

20. The fusion protein according to of claim 2, wherein each TNFalpha monomer is a human TNFalpha.

21. The fusion protein according to claim 5, wherein the target molecule is an extradomain B (ED-B) of fibronectin.

22. The pharmaceutical composition of claim 12, wherein the one or more chemotherapeutic agents are selected from the group consisting of alkylating agents, intercalating agents, platinum analogs, antibiotics, taxanes, anti-metabolites, mitosis inhibitors, topoisomerase inhibitors, and radiopharmaceuticals.

23. The method of claim 15, wherein the nucleic acid molecule further comprises a polynucleotide sequence encoding a peptide linker positioned between and in frame with the polynucleotide sequence encoding the modified dimeric ubiquitin protein of step (e) and the polynucleotide sequence encoding a biologically active single chain TNFalpha molecule such that the nucleic acid molecule encodes a fusion protein comprising the modified dimeric ubiquitin protein of step (e) linked to the biologically active single chain TNFalpha molecule via the peptide linker.


SEQUENCE LISTING (29 sequences)

SEQ ID NO: 1 - protein, 76 aa, Homo sapiens [sequence omitted]

SEQ ID NO: 2 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 3 - protein, 3 aa, Artificial Sequence
  Artificially synthesized linker sequence
GIG

SEQ ID NO: 4 - protein, 5 aa, Artificial Sequence
  Artificially synthesized linker sequence
SGGGG

SEQ ID NO: 5 - protein, 7 aa, Artificial Sequence
  Artificially synthesized linker sequence
SGGGGIG

SEQ ID NO: 6 - protein, 12 aa, Artificial Sequence
  Artificially synthesized linker sequence
SGGGGSGGGGIG

SEQ ID NO: 7 - protein, 10 aa, Artificial Sequence
  Artificially synthesized linker sequence
SGGGGSGGGG

SEQ ID NO: 8 - protein, 15 aa, Artificial Sequence
  Artificially synthesized linker sequence
GGGGSGGGGSGGGGS

SEQ ID NO: 9 - protein, 12 aa, Artificial Sequence
  Artificially synthesized linker sequence
GGGSGGGSGGGS

SEQ ID NO: 10 - protein, 76 aa, Artificial Sequence
  ubiquitin start sequence (F45W, G75A, G76A)
MQIFVKTLTGKTITLEVEPSDTIENVKAKIQDKEGIPPDQQRLIWAGKQLEDGRTLSDYNIQKESTLHLVLRLRAA

SEQ ID NO: 11 - protein, 155 aa, Artificial Sequence
  Ub2; dimer of unmodified ubiquitin start sequence with GIG linker
MQIFVKTLTGKTITLEVEPSDTIENVKAKIQDKEGIPPDQQRLIWAGKQLEDGRTLSDYNIQKESTLHLVLRLRAAGIGMQIFVKTLTGKTITLEVEPSDTIENVKAKIQDKEGIPPDQQRLIWAGKQLEDGRTLSDYNIQKESTLHLVLRLRAA

SEQ ID NO: 12 - protein, 155 aa, Artificial Sequence
  anti-ED-B affilin(R) 54646
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 13 - protein, 157 aa, Homo sapiens [sequence omitted]

SEQ ID NO: 14 - protein, 156 aa, Mus musculus
LRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 15 - protein, 156 aa, Rattus norvegicus
LRSSSQNSSDKPVVHVVANHQAEEQLEWLSQRANALLANGMDLKDNQLVVPADGLYLIYSQVLFKGQGCPDYVLLTHTVSRFATSYQEKVSLLSAIKSPCPKDTPEGAELKPWYEPMYLGGVSQLEKGDLLSAEVNLPKYLDITESGQVYFGVIAL

SEQ ID NO: 16 - protein, 662 aa, Artificial Sequence
  64179, fusion protein of Ub2 and scTNFalpha, murine
MQIFVKTLTGKTITLEVEPSDTIENVKAKIQDKEGIPPDQQRLIWAGKQLEDGRTLSDYNIQKESTLHLVLRLRAAGIGMQIFVKTLTGKTITLEVEPSDTIENVKAKIQDKEGIPPDQQRLIWAGKQLEDGRTLSDYNIQKESTLHLVLRLRAAGGGGSGGGGSGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 17 - protein, 662 aa, Artificial Sequence
  64177, fusion protein of 54646 and scTNFalpha, murine
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 18 - protein, 665 aa, Artificial Sequence
  75808, fusion protein of 54646 and scTNFalpha, human
MTIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 19 - protein, 662 aa, Artificial Sequence
  83563, mutein (T2V) of fusion protein 64177
MVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 20 - protein, 665 aa, Artificial Sequence
  h83563, mutein (T2V) of fusion protein 75808
MVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 21 - protein, 662 aa, Artificial Sequence
  83564, mutein (T2R/F63P) of fusion protein 64177
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIALGGGSGGGSGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 22 - protein, 665 aa, Artificial Sequence
  h83564, mutein (T2R/F63P) of fusion protein 75808
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAAGGGGSGGGGSGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 23 - protein, 155 aa, Artificial Sequence
  anti-ED-B Affilin(R), mutein (T2V) of 54646
MVIWVHTLTGKTITLEVEPSDTIENVKAKIQDKEGIPPDQQRLIWAGKQLEDGRTLSDYNINFKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 24 - protein, 155 aa, Artificial Sequence
  anti-ED-B Affilin(R), mutein (T2R/F63P) of 54646
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 25 - protein, 158 aa, Artificial Sequence
  consensus sequence of TNFalpha
MLRSSSQNSSDKPVAHVVANHQAEEQLEWLSQRANALLANGMDLKDNQLVVPADGLYLIYSQVLFKGQGCPSDYVLLTHTVSRFAISYQEKVNLLSAIKSPCPKDTPEGAELKPWYEPIYLGGVFQLEKGDQLSAEVNLPKYLDFAESGQVYFGVIAL

SEQ ID NO: 26 - protein, 17 aa, Artificial Sequence
  Artificially synthesized linker sequence
SGGGGGSGGGGSGGGGS

SEQ ID NO: 27 - protein, 7 aa, Artificial Sequence
  Artificially synthesized linker sequence
SGGGGGS

SEQ ID NO: 28 - protein, 5 aa, Artificial Sequence
  Artificially synthesized linker sequence
GGGGS

SEQ ID NO: 29 - protein, 15 aa, Artificial Sequence
  Artificially synthesized linker sequence
SSSSGSSSSGSSSSG